(19) United States
(12) Patent Application Publication
(10) Pub. No.: US 2025/0305023 A1
(43) Pub. Date: Oct. 2, 2025

(54) MEASUREMENT OF PERMEATION OF SMALL MOLECULES INTO GRAM NEGATIVE BACTERIA

(71) Applicant: University of Virginia Patent Foundation, Charlottesville, VA (US)

(72) Inventor: Marcos M. Pires, Crozet, VA (US)

(21) Appl. No.: 18/862,887

(22) PCT Filed: May 4, 2023
(86) PCT No.: PCT/US2023/066596

§ 371 Date: Nov. 4, 2024

Related U.S. Application Data
(60) Provisional application No. 63/364,235, filed on May 5, 2022.

(51) Int. Cl. C12Q 1/20

(52) U.S. Cl. CPC C12Q 1/20

(57) ABSTRACT

Disclosed herein are processes term Bacterial Chloro-Alkane Penetration Assay (BaCAPA) and Bacterial Azide Permeability Assay (BAPA) The processes employ a genetically encoded protein called HaloTag to measure the uptake and accumulation of molecules into Gram-negative bacteria. The processes aqre useful in assessing the permeation of molecules within the phagocytes of macrophages, and they effectively report on the accumulation of molecules into bacterial cells, thereby identifying potential antibiotic drugs.

This application claims the benefit of priority to U.S. Provisional Patent Application No. 63/364,235 filed on May 5, 2022, which application is incorporated in its entirety as if fully set forth herein.

### GOVERNMENT LICENSE RIGHTS

This invention was made with government support under grant GM124893-01 awarded by the National Institutes of Health. The government has certain rights in the invention

### BACKGROUND

The rise in incidence of drug-resistant bacterial infections poses a tremendous challenge for healthcare systems throughout the world.¹⁻³ While recent efforts have led to the discovery of new therapeutic leads with promising clinical potential,⁴⁻¹⁰ there is a continued need to strengthen the antibiotic pipeline and to find antibiotics with narrow spectrum activities to reduce off target impact on gut commensal bacteria. Most antibiotics must enter the bacterial cell to impart their biological effects, which includes permeating through a lipid bilayer. For Gram-negative bacteria, there is an additional challenge due to the presence of an asymmetrical bilayer known as the outer membrane (OM).¹¹⁻¹² For some agents (e.g., β-lactam antibiotics), permeation through OM-anchored porins can potentially provide an access pathway to the periplasm.¹³ However, many of the most potent antibiotics have cytosolic targets, and, therefore to be effective they must also permeate through the inner plasma membrane. Additionally, a reduction in the amount of antibiotics accumulating in Gram-negative bacteria can be further modulated by the active removal of drugs that reach the periplasm by efflux pumps that recognize broad structural motifs.¹⁴⁻¹⁵

Due to a lack of effective treatment options, projections indicate that by 2050, more people will die from antibacterial resistant infections than cancer (Tackling Drug-Resistant Infections Globally—Final Report and Recommendations, Review on Antimicrobial Resistance (2016). The ability to treat infections will be increasingly compromised as cases of drug-resistant bacteria become more prevalent (Bush K, Courvalin P, Dantas G, et al. Tackling antibiotic resistance. Nat Rev Microbiol. December 2011; 9(12):894-6; Spellberg B, Shlaes D. Prioritized Current Unmet Needs for Antibacterial Therapies. Clin Pharmacol Ther. August 2014; 96(2):151-153). Moreover, the lack of effective antibiotics erodes other significant medical gains (e.g., organ transplants, invasive surgeries, and cancer chemotherapy), which have been critically dependent on antibiotics for their successes. As noted above, a primary barrier to the discovery of new agents has been the general lack of permeability of small molecules into Gram-negative bacteria.

The increased prevalence of multidrug resistant Gram-negative bacterial infections is alarming (Marston H D, Dixon D M, Knisely J M, Palmore T N, Fauci A S. Antimicrobial Resistance. JAMA. Sep. 20 2016; 316(11):1193-1204). The CDC issued a report in 2019 that categorized bacterial pathogens based on their threat levels. The list of pathogens considered “urgent threats” was primarily populated by antibiotic-resistant Gram-negative bacteria and included Escherichia coli (E. coli). Lack of treatment options could usher in a post-antibiotic era, meaning that routine infections can become lethal and standard invasive medical procedures will carry much higher levels of risk. These reasons underscore the significance of developing new strategies to address Gram-negative pathogens.

There is wide agreement in the community that antibiotic drug discovery has been hampered by the lack of robust and widely adoptable tools to measure the accumulation of molecules into bacteria (Six D A, Krucker T, Leeds J A. Advances and challenges in bacterial compound accumulation assays for drug discovery. Curr Opin Chem Biol. June 2018; 44:9-15; Ferreira R J, Kasson P M. Antibiotic Uptake Across Gram-Negative Outer Membranes: Better Predictions Towards Better Antibiotics. ACS Infect Dis. Dec. 13 2019; 5(12):2096-2104; Hancock R E. The bacterial outer membrane as a drug barrier. Trends Microbiol. January 1997; 5(1):37-42; Cama J, Henney A M, Winterhalter M. Breaching the Barrier: Quantifying Antibiotic Permeability across Gram-negative Bacterial Membranes. J Mol Biol. 08 23 2019; 431(18):3531-3546; Zhao S, Adamiak J W, Bonifay V, Mehla J, Zgurskaya H I, Tan D S. Defining new chemical space for drug penetration into Gram-negative bacteria. Nat Chem Biol. December 2020; 16(12):1293-1302; Prajapati J D, Kleinekathofer U, Winterhalter M. How to Enter a Bacterium: Bacterial Porins and the Permeation of Antibiotics. Chem Rev. May 12 2021; 121(9):5158-5192; Delcour A H. Outer membrane permeability and antibiotic resistance. Biochim Biophys Acta. May 2009; 1794(5):808-16; Zgurskaya H I, Lopez C A, Gnanakaran S. Permeability Barrier of Gram-Negative Cell Envelopes and Approaches To Bypass It. ACS Infect Dis. 2015; 1(11):512-522; Vergalli J, Bodrenko I V, Masi M, et al. Porins and small-molecule translocation across the outer membrane of Gram-negative bacteria. Nat Rev Microbiol. March 2020; 18(3):164-176; Bolla J M, Alibert-Franco S, Handzlik J, et al. Strategies for bypassing the membrane barrier in multidrug resistant Gram-negative bacteria. FEBS Lett. Jun. 6 2011; 585(11):1682-90).

The golden age of antibiotics leveraged naturally abundant small molecules that were readily identified using traditional methods. Since then, however, it has proven to be much more difficult to use these methods to mine for new antibiotics. The next phase of antibiotic drug discovery could potentially leverage the wealth of existing proteomics, genomics, and metabolomics data to design small molecule agents that are potent and of high specificity. To accomplish this, the field needs guiding principles describing the molecular determinants of small molecule permeation into Gram-negative bacterial cells akin to Lipinski's rules of 5 (Ro5).

Gram-negative pathogens are intrinsically less susceptible to antibiotics due to the unique composition of their cell walls. More specifically, in addition to the canonical plasma membrane, Gram-negative bacteria display a second barrier. This asymmetric outer membrane has structural features that are well-adopted to significantly diminish the permeation of small molecules into the periplasmic space. In particular, the impermeability of the outer membrane poses a significant challenge to target-based screening efforts. While several potent enzyme inhibitors have been discovered in screening campaigns, a large majority of these fail to reach their target due to poor outer membrane permeation.

The types of molecules that effectively permeate the outer membrane typically fall into two categories (Brown D G, May-Dracka T L, Gagnon M M, Tommasi R. Trends and exceptions of physical properties on antibacterial activity for Gram-positive and Gram-negative pathogens. J Med Chem. Dec. 11 2014; 57(23):10144-61; O'Shea R, Moser H E. Physicochemical properties of antibacterial compounds: implications for drug discovery. J Med Chem. May 22 2008; 51(10):2871-8). First, a subset of molecules fit a narrow range of physiochemical properties (e.g., molecular weight, number of hydrogen bonds, etc.) that permits passive diffusion across the outer membrane. Second, others are hydrophilic molecules (e.g., β-lactam antibiotics) that influx through porins imbedded within the outer membrane to avoid the non-polar regions of this bilayer. Even so, there remains a paucity of well-defined parameters to guide the iterative redesign of non-permeators into active agents. Molecular descriptors have not been well defined because there is a lack of easily adoptable tools to measure the accumulation of small molecules in bacteria.

In the absence of facile methods to measure uptake into bacterial cells, microbiologists have typically resorted to use of antimicrobial activity (minimum inhibitory activity, MIC) as an approximation for drug accumulation. This approach has serious drawbacks because drug accumulation can be inherently independent from drug potency. Moreover, antivirulent agents or potentiators often lack antimicrobial activities on their own, and, therefore, cannot be subjected to MIC analyses.

Despite the fact that drug permeation is well recognized as a major bottleneck for antibiotic efficacy and directly tied to drug discovery efforts (O'Shea (2008); Kojima S, Nikaido H. Permeation rates of penicillins indicate that Escherichia coli porins function principally as nonspecific channels. Proc Natl Acad Sci USA. Jul. 9 2013; 110(28):E2629-34), there are few methods developed to measure the accumulation of small molecules in Gram-negative bacteria (June C M, Vaughan R M, Ulberg L S, Bonomo R A, Witucki L A, Leonard D A. A fluorescent carbapenem for structure function studies of penicillin-binding proteins, beta-lactamases, and beta-lactam sensors. Anal Biochem. Oct. 15 2014; 463:70-4). As such, gaps remain in our fundamental understanding of the molecular determinants of permeability. Direct methods of measuring drug uptake and retention can be extremely valuable in providing insight into drug accumulation profiles. The Hergenrother group¹⁹ adopted a protocol²⁰ that uses liquid chromatography with tandem mass spectrometry (LC-MS/MS) to expand the analysis of drug permeation of over 180 diverse molecules in Escherichia coli (E. coli). A principal finding from these investigations was that primary amines as a functional group are privileged moieties in promoting accumulation in Gram-negative bacteria. The efforts used conventional LC-MS/MS methods with each individual molecule requiring its unique calibration curve for quantification, but still the total range of chemical space evaluated was relatively modest. While informative in its one-off way, the 188-molecule screening campaign by a large consortium does not cover the scope necessary to broadly provide structural determinants of molecule permeation in Gram-negative bacteria. Indeed, since its description in 2017 there has not been a similarly large-scale effort. This traditional method continues to be low throughput for every other laboratory interested in assessing cellular uptake in bacteria. More recent studies leveraged biorthogonal reactions to probe the accumulation to distinct compartments within E. coli (Cama J, Bajaj H, Pagliara S, et al. Quantification of Fluoroquinolone Uptake through the Outer Membrane Channel OmpF of Escherichia coli. J Am Chem Soc. Nov. 4 2015; 137(43):13836-43; Ghai I, Winterhalter M, Wagner R. Probing transport of charged beta-lactamase inhibitors through OmpC, a membrane channel from E. coli. Biochem Biophys Res Commun. Feb. 26 2017; 484(1):51-55).

### SUMMARY

The present disclosure provides, in various embodiments, a process for determining whether a small molecule is a potential antibiotic drug, comprising:
(a) contacting the small molecule that has been linked to a chloroalkane moiety with a bacteria that expresses a mutant form of bacterial haloalkane dehalogenase to yield a first population of treated bacteria;
(b) contacting the first population of treated bacteria with a fluorophore that is linked to a chloroalkane moiety to produce a second population of treated bacteria;
(c) detecting the fluorescence signal of the second population of treated bacteria; and
(d) determining that a low fluorescence signal relative to background fluorescence correlates to a conclusion of high accumulation of the small molecule within the bacteria, and that a high fluorescence signal relative to background fluorescence correlates to a conclusion of low accumulation of the small molecule within the bacteria.

In additional embodiments, the present disclosure provides a process for determining whether a small molecule is a potential antibiotic drug. The process comprises:
(a1) contacting a bacteria that expresses a mutant form of bacterial haloalkane dehalogenase with a reagent of the general formula (I) to yield a tagged population of bacteria:

[strained alkyne]-(linker)-(alk-Cl) (I)
wherein
[strained alkyne] is a moiety containing a strained alkyne functional group;
(linker) is selected from a bond, amino, amido, carboxyl, C₁-C₆-ester, C₁-C₆-alkoxy, linear and cyclic boronic esters, 3- to 8-membered heterocycloalkyl (wherein 1 to 3 heteroatoms are selected from N, O, and S), C₃-C₈-cycloalkyl optionally fused to 1 or 2 C₆-C₁₀-aryl, C₆-C₁₀-aryl, 5- to 10-membered heteroaryl (wherein 1 to 4 heteroatoms are selected from N, O, and S), succinimidyl, C₁-C₆-alkyl optionally interrupted by one or more of —C(O)— and —N(H)—, and combinations thereof; and
(alk-Cl) is a straight or branched C₁-C₂₀-chloroalkyl optionally interrupted by 1 to 6 oxygen atoms;
(b1) contacting the tagged population of bacteria with the small molecule that has been linked to an azide moiety to yield a first population of treated bacteria;
(c1) contacting the first population of treated bacteria with a fluorophore that is linked to an azide moiety to yield a second population of treated bacteria;
(d1) detecting a fluorescence signal of the second population of treated bacteria; and
(e1) determining that a low fluorescence signal relative to background fluorescence correlates to a conclusion of high accumulation of the small molecule within the bacteria, and that a high fluorescence signal relative to background fluorescence correlates to a conclusion of low accumulation of the small molecule within the bacteria.

### BRIEF DESCRIPTION OF THE DRAWINGS

FIGS. 1A and 1B. (A) Schematic representation of BaCAPA. Bacterial cells expressing HaloTag will covalently react with molecules of interest, thus reducing the number of sites available to react with the fluorophore. (B) HaloTag reaction to illustrative chloroalkane linked molecule.

FIG. 2A-2F. (A) Schematic representation of reaction of a fluorophore-linked to chloroalkane with HaloTag inside a bacterial cell. (B) Flow cytometry analysis of K-12 E. coli (rough) treated with various concentration of IPTG, followed by an incubation period with 5 μM of R110cl. (C) Flow cytometry analysis of E. coli treated with either 5 μM of R110cl or COMcl. (D) Flow cytometry analysis of E. coli treated with various concentrations of R110cl in the presence and absence of IPTG induction. Data are represented as mean+/−SD (n=3). P-values were determined by a two-tailed t-test (* denotes a p-value<0.05, **<0.01, ***<0.001, ns=not significant). (E) Coomassie (left) and fluorescence images of SDS-PAGE gel of whole cellular extracts of E. coli treated with 5 mM of R110cl in the presence and absence of IPTG induction. (F) Confocal microscopy analysis of E. coli induced with IPTG and treated with 5 mM of R110cl. Cells were fixed with 2% formaldehyde, deposited onto a pre-cooled 1% (w/v) agarose pad placed on a glass microscope slide, and covered with a micro cover glass. Images were collected with a Zeiss 880/990 multiphoton Airyscan microscopy system (60× oil-immersion lens). Scale bar=2 μm.

FIG. 3A-3G. Flow cytometry analysis of E. coli treated with various concentrations of PMBN (A) or liproxstatin (B), followed by a treatment with 5 μM of R110cl in the presence and absence of IPTG induction. Data are represented as mean+/−SD (n=3). P-values were determined by a two-tailed t-test (* denotes a p-value<0.05, **<0.01, ***<0.001, ns=not significant). (C) E. coli cells were treated with 10 mM of PMBN. (D) E. coli was treated with 5 mM of R110cl in the presence and absence of IPTG induction. After washing cells with PBS (1×), cellular fluorescence was measured using a plate reader (excitation=480 nm (40 nm), emission=520 nm (40 nm)). (E) Flow cytometry analysis of E. coli (ATCC 25922) treated with 5 μM of R110cl in the presence and absence of IPTG induction. Data are represented as mean+/−SD (n=3). P-values were determined by a two-tailed t-test (* denotes a p-value<0.05, **<0.01, ***<0.001, ns=not significant). (F) Flow cytometry analysis of E. coli (Lemo) carrying a plasmid of HaloTag fused with DsbA signal peptide treated with 5 mM of R110cl in the presence and absence of IPTG induction. Data are represented as mean+/−SD (n=3). P-values were determined by a two-tailed t-test (* denotes a p-value<0.05, **<0.01, ***<0.001, ns=not significant). (G) Confocal microscopy analysis of E. coli (Lemo) carrying a plasmid of HaloTag fused with DsbA signal peptide treated with 5 mM of R110cl in the presence of IPTG induction. Cells were fixed with 2% formaldehyde, deposited onto a pre-cooled 1% (w/v) agarose pad placed on a glass microscope slide, and covered with a micro cover glass. Images were collected with a Zeiss 880/990 multiphoton Airyscan microscopy system (60× oil-immersion lens). Scale bar=2 μm.

FIGS. 4A and 4B. (4A) Flow cytometry analysis of E. coli treated with various concentrations of molecules 2-5, followed by a treatment with 5 μM of R110cl in the presence and absence of IPTG induction. Data are represented as mean+/−SD (n=3). (4B) Flow cytometry analysis of E. coli treated with increasing concentrations of the amine ligand, followed by treatment with 5 μM of R110cl in the presence of IPTG induction. Data are represented as mean+/−SD (n=3).

FIG. 5. Flow cytometry analysis of E. coli treated with various concentrations of molecules 6-8, followed by a treatment with 5 μM of R110cl in the presence and absence of IPTG induction. Data are represented as mean+/−SD (n=3).

FIG. 6A-FIG. 6D. (A) Schematic representation of BaCAPA of bacteria inside mammalian cells. (B) Flow cytometry analysis of J774 cells alone, or J774 cells phagocytosed E. coli in the presence and absence of IPTG induction treated with 5 μM of R110cl. Data are represented as mean+/−SD (n=3). P-values were determined by a two-tailed t-test (* denotes a p-value<0.05, **<0.01, ***<0.001, ns=not significant). (C) Confocal microscopy images of J774 macrophages with phagocytosed E. coli that were treated with 5 μM of R110cl. Cells were fixed with formaldehyde and treated with tetramethyl-rhodamine-tagged WTA (5 μg/mL) for 30 min. Shown is the overlay of the channels corresponding to rhodamine 110 and tetramethyl-rhodamine. Scale bar=10 μm. (D). Flow cytometry analysis of E. coli treated with 5 μM of R110cl in the presence and absence of IPTG induction. The same cells were used for the analysis of labeling bacteria in macrophages. Data are represented as mean+/−SD (n=3). P-values were determined by a two-tailed t-test (* denotes a p-value<0.05, **<0.01, ***<0.001, ns=not significant).

FIG. 7. Illustrative process of BAPA for azide-tagging.

FIGS. 8A-8C. IPTG (A) and R110-N3 (B) concentration response of BAPA in E. coli. (C) BAPA test with commercially available 24 azide-tagged small molecules.

FIG. 9. Chemical structures of synthesized azide-tagged antibiotic compounds.

FIG. 10. Analysis of ˜1200 azide-tagged compounds using BAPA in E. coli.

### DETAILED DESCRIPTION

The present disclosure addresses various shortcomings in the art by providing methodology to quantitatively measure the accumulation of small molecules into Gram-negative bacteria. Most methods known in the art for measuring compound uptake do not establish the extent of permeation across subcellular compartments. In instances when they do, the method (fractionalization) is not compatible with higher-throughput analyses. Moreover, the known methods have been limited in the kinds of molecules for which bacterial penetration can be assessed. In contrast, an advantage of the present disclosure is premised, in part, upon the use of biorthogonal tags to establish molecular determinants of Gram-negative permeability. Further, the methods described herein, in contrast to known methods, are amenable to significantly larger scale of analysis in drug penetration in Gram-negative bacteria, e.g., from about 180 to over 5000, respectively. This is not an incremental increase but instead, a leap forward in terms of the chemical space that is probed and described. Another advantage of the methods of the present disclosure resides in their translation to essentially all Gram-negative pathogens of interest.

### Definitions

Unless defined otherwise, all technical and scientific terms used herein have the same meaning as commonly understood by one of ordinary skill in the art to which this disclosure pertains.

As used herein and in the appended claims, the singular forms “a,” “and,” and “the” include plural referents unless the context clearly dictates otherwise. Thus, for example, reference to “an agent” includes a plurality of such agents, and reference to “the cell” includes reference to one or more cells (or to a plurality of cells) and equivalents thereof known to those skilled in the art, and so forth.

When ranges are used herein for physical properties, such as molecular weight, or chemical properties, such as chemical formulae, all combinations and subcombinations of ranges and specific embodiments therein are intended to be included. The term “about” when referring to a number or a numerical range means that the number or numerical range referred to is an approximation within experimental variability (or within statistical experimental error), and thus the number or numerical range, in some instances, will vary between 1% and 15% of the stated number or numerical range.

In the present disclosure, the number of atoms of a particular element in a substituent group is generally given as a range, e.g., an alkyl group containing from 1 to 4 carbon atoms or C₁₋₄ alkyl. Reference to such a range is intended to include specific references to groups having each of the integer number of atoms within the specified range. For example, an alkyl group from 1 to 4 carbon atoms includes each of C₁, C₂, C₃, and C₄. A C₁₋₁₂ heteroalkyl, for example, includes from 1 to 12 carbon atoms in addition to one or more heteroatoms. Other numbers of atoms and other types of atoms may be indicated in a similar manner.

The term “comprising” (and related terms such as “comprise” or “comprises” or “having” or “including”) is not intended to exclude that in other certain embodiments, for example, an embodiment of any composition of matter, composition, method, or process, or the like, described herein, “consist of” or “consist essentially of” the described features.

“Alkyl” refers to straight or branched chain hydrocarbyl including from 1 to about 20 carbon atoms. For instance, an alkyl can have from 1 to 10 carbon atoms or 1 to 6 carbon atoms. Exemplary alkyl includes straight chain alkyl groups such as methyl, ethyl, propyl, butyl, pentyl, hexyl, heptyl, octyl, nonyl, decyl, undecyl, dodecyl, and the like, and also includes branched chain isomers of straight chain alkyl groups, for example without limitation, —CH(CH₃)₂, —CH(CH₃)(CH₂CH₃), —CH(CH₂CH₃)₂, —C(CH₃)₃, —C(CH₂CH₃)₃, —CH₂ CH(CH₃)₂, —CH₂CH(CH₃)(CH₂CH₃), —CH₂CH(CH₂CH₃)₂, —CH₂C(CH₃)₃, —CH₂C(CH₂CH₃)₃, —CH(CH₃) CH(CH₃)(CH₂CH₃), —CH₂CH₂CH(CH₃)₂, —CH₂CH₂CH(CH₃)(CH₂CH₃), —CH₂CH₂C H(CH₂CH₃)₂, —CH₂CH₂C(CH₃)₃, —CH₂CH₂C(CH₂CH₃)₃, —CH(CH₃)CH₂CH(CH₃)₂, —CH(CH₃) CH(CH₃) CH(CH₃)₂, and the like. Thus, alkyl groups include primary alkyl groups, secondary alkyl groups, and tertiary alkyl groups. An alkyl group can be unsubstituted or optionally substituted with one or more substituents as described herein, such as halogen(s), for example.

Each of the terms “halogen,” “halide,” and “halo” refers to —F or fluoro, —Cl or chloro, —Br or bromo, or —I or iodo.

The term “alkenyl” refers to straight or branched chain hydrocarbyl groups including from 2 to about 20 carbon atoms having 1-3, 1-2, or at least one carbon to carbon double bond. An alkenyl group can be unsubstituted or optionally substituted with one or more substituents as described herein.

“Alkyne or “alkynyl” refers to a straight or branched chain unsaturated hydrocarbon having the indicated number of carbon atoms and at least one triple bond. Examples of a (C₂-C₈) alkynyl group include, but are not limited to, acetylene, propyne, 1-butyne, 2-butyne, 1-pentyne, 2-pentyne, 1-hexyne, 2-hexyne, 3-hexyne, 1-heptyne, 2-heptyne, 3-heptyne, 1-octyne, 2-octyne, 3-octyne and 4-octyne. An alkynyl group can be unsubstituted or optionally substituted with one or more substituents as described herein.

The term “cycloalkyl” refers to a saturated monocyclic, bicyclic, tricyclic, or polycyclic, 3- to 14-membered ring system, such as a C₃-C₈-cycloalkyl. The cycloalkyl may be attached via any atom. Representative examples of cycloalkyl include, but are not limited to cyclopropyl, cyclobutyl, cyclopentyl, and cyclohexyl. A cycloalkyl group can be unsubstituted or optionally substituted with one or more substituents as described herein.

“Aryl” (Ar) when used alone or as part of another term means a carbocyclic aromatic group whether or not fused having the number of carbon atoms designated or if no number is designated, up to 14 carbon atoms, such as a C₆-C₁₀-aryl or C₆-C₁₄-aryl. In embodiments, an Ar may be characterized by an aromatic group having a ring system comprised of carbon atoms with conjugated π electrons (e.g., phenyl). The term includes aryl groups having from 6 to 12 carbon atoms. Aryl groups may optionally include monocyclic, bicyclic, or tricyclic rings, in which each ring has five or six members. Examples of aryl groups include phenyl, naphthyl, biphenyl, phenanthrenyl, naphthacenyl, and the like (see e.g. Lang's Handbook of Chemistry (Dean, J. A., ed) 13^{th} ed. Table 7-2 [1985]). “Aryl” also contemplates an aryl ring that is part of a fused polycyclic system, such as aryl fused to cycloalkyl as defined herein. An exemplary aryl is phenyl. An aryl group can be unsubstituted or optionally substituted with one or more substituents as described herein.

The term “heteroatom” refers to N, O, and S. Compounds of the present disclosure that contain N or S atoms can be optionally oxidized to the corresponding N-oxide, sulfoxide, or sulfone compounds.

“Heteroaryl,” alone or in combination with any other moiety described herein, is a monocyclic aromatic ring structure containing 5 to 10, such as 5 or 6 ring atoms, or a bicyclic aromatic group having 8 to 10 atoms, containing one or more, such as 1-4, 1-3, or 1-2, heteroatoms independently selected from the group consisting of O, S, and N. Heteroaryl is also intended to include oxidized S or N, such as sulfinyl, sulfonyl and N-oxide of a tertiary ring nitrogen. A carbon or heteroatom is the point of attachment of the heteroaryl ring structure such that a stable compound is produced. Examples of heteroaryl groups include, but are not limited to, pyridinyl, pyridazinyl, pyrazinyl, quinaoxalyl, indolizinyl, benzo[b]thienyl, quinazolinyl, purinyl, indolyl, quinolinyl, pyrimidinyl, pyrrolyl, pyrazolyl, oxazolyl, thiazolyl, thienyl, isoxazolyl, oxathiadiazolyl, isothiazolyl, tetrazolyl, imidazolyl, triazolyl, furanyl, benzofuryl, and indolyl. A heteroaryl group can be unsubstituted or optionally substituted with one or more substituents as described herein.

“Heterocycloalkyl” is a saturated or partially unsaturated non-aromatic monocyclic, bicyclic, tricyclic or polycyclic ring system that has from 3 to 14, such as 3 to 6, atoms in which 1 to 3 carbon atoms in the ring are replaced by heteroatoms of O, S or N. The ring heteroatoms can also include oxidized S or N, such as sulfinyl, sulfonyl, and N-oxides of a tertiary ring nitrogen. A heterocycloalkyl can be fused to another ring system, such as with an aryl or heteroaryl of 5-6 ring members. The point of attachment of the heterocycloalkyl ring is at a carbon or heteroatom such that a stable ring is retained. Examples of heterocycloalkyl groups include without limitation morpholino, tetrahydrofuranyl, dihydropyridinyl, piperidinyl, pyrrolidinyl, piperazinyl, dihydrobenzofuryl, and dihydroindolyl. A heterocycloalkyl group can be unsubstituted or optionally substituted with one or more substituents as described herein.

Compounds described herein can exist in various isomeric forms, including configurational, geometric, and conformational isomers, including, for example, cis- or trans-conformations. The compounds may also exist in one or more tautomeric forms, including both single tautomers and mixtures of tautomers. The term “isomer” is intended to encompass all isomeric forms of a compound of this disclosure, including tautomeric forms of the compound. The compounds of the present disclosure may also exist in open-chain or cyclized forms. In some cases, one or more of the cyclized forms may result from the loss of water. The specific composition of the open-chain and cyclized forms may be dependent on how the compound is isolated, stored or administered. For example, the compound may exist primarily in an open-chained form under acidic conditions but cyclize under neutral conditions. All forms are included in the disclosure.

Some compounds described herein can have asymmetric centers and therefore exist in different enantiomeric and diastereomeric forms. A compound as described herein can be in the form of an optical isomer or a diastereomer. Accordingly, the disclosure encompasses compounds and their uses as described herein in the form of their optical isomers, diastereoisomers and mixtures thereof, including a racemic mixture. Optical isomers of the compounds of the disclosure can be obtained by known techniques such as asymmetric synthesis, chiral chromatography, simulated moving bed technology or via chemical separation of stereoisomers through the employment of optically active resolving agents.

Unless otherwise indicated, the term “stereoisomer” means one stereoisomer of a compound that is substantially free of other stereoisomers of that compound. Thus, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, for example greater than about 90% by weight of one stereoisomer of the compound and less than about 10% by weight of the other stereoisomers of the compound, or greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound, or greater than about 99% by weight of one stereoisomer of the compound and less than about 1% by weight of the other stereoisomers of the compound. The stereoisomer as described above can be viewed as composition comprising two stereoisomers that are present in their respective weight percentages described herein.

If there is a discrepancy between a depicted structure and a name given to that structure, then the depicted structure controls. Additionally, if the stereochemistry of a structure or a portion of a structure is not indicated with, for example, bold or dashed lines, the structure or portion of the structure is to be interpreted as encompassing all stereoisomers of it. In some cases, however, where more than one chiral center exists, the structures and names may be represented as single enantiomers to help describe the relative stereochemistry. Those skilled in the art of organic synthesis will know if the compounds are prepared as single enantiomers from the methods used to prepare them.

As used herein, and unless otherwise specified to the contrary, the term “compound” is inclusive in that it encompasses a compound or a pharmaceutically acceptable salt, stereoisomer, isotopologue, and/or tautomer thereof. Thus, for instance, a compound includes a pharmaceutically acceptable salt of a tautomer of the compound. Similarly, a compound of includes a pharmaceutically acceptable salt of an isotopologue of the compound.

### Bacterial ChloroAlkane Penetration Assay (BaCAPA)

In various embodiments, the present disclosure provides a process for determining whether a small molecule is a potential antibiotic drug, comprising:
(a) contacting the small molecule that has been linked to a chloroalkane moiety with a bacteria that expresses a mutant form of bacterial haloalkane dehalogenase to yield a first population of treated bacteria;
(b) contacting the first population of treated bacteria with a fluorophore that is linked to a chloroalkane moiety to produce a second population of treated bacteria;
(c) detecting the fluorescence signal of the second population of treated bacteria; and
(d) determining that a low fluorescence signal relative to background fluorescence correlates to a conclusion of high accumulation of the small molecule within the bacteria, and that a high fluorescence signal relative to background fluorescence correlates to a conclusion of low accumulation of the small molecule within the bacteria.

In some embodiments, the fluorophore is a rhodamine dye or coumarin. In one embodiment, the fluorophore is a rhodamine dye.

In additional embodiments, the chloroalkane moiety linked to the fluorophore is of the formula:

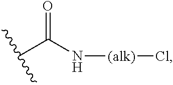

wherein alk is a straight or branched C₁-C₂₀-alkyl optionally interrupted by 1 to 6 oxygen atoms.

In additional embodiments, the chloroalkane moiety linked to the small molecule is of the formula:

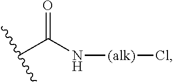

wherein alk is a straight or branched C₁-C₂₀-alkyl optionally interrupted by 1 to 6 oxygen atoms.

In further embodiments, the bacteria is E. coli.

In still additional embodiments, the bacteria reside inside a mammalian cell. An illustrative mammalian cell is a macrophage.

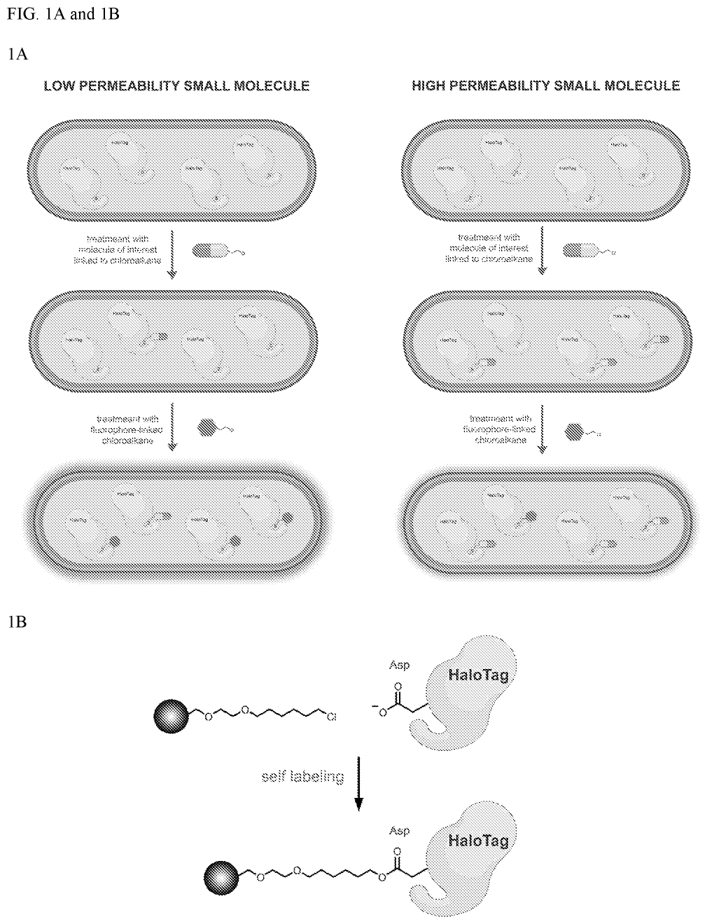

The process is useful as an assay method that is adapted from the chloroalkane penetration assay (CAPA; Kascakova S, Maigre L, Chevalier J, Refregiers M, Pages J M. Antibiotic transport in resistant bacteria: synchrotron UV fluorescence microscopy to determine antibiotic accumulation with single cell resolution. PLOS One. 2012; 7(6)), which is measures the accumulation of molecules into mammalian cells. This method relies on the genetically encoded HaloTag²⁴, a mutant form of a bacterial haloalkane dehalogenase that facilitates covalent bond formation with chloroalkane-linked ligands (FIG. 1). Previous efforts utilizing the HaloTag protein within bacteria focused on the visualization of protein fusions²⁵⁻²⁶ and the role of a cationic peptide on membrane permeabilization.²⁷ Bacterial Chloroalkane Penetration Assay (BaCAPA) measures the apparent accumulation of molecules into bacterial cells. The assay is based on the expression of the HaloTag protein inside bacterial cells, which are exposed to molecules of interest linked to a chloroalkane tag. If the molecules reach the HaloTag proteins inside the bacteria, they will be covalently bound to the protein. This pulse step is followed by a chase step with a fluorophore-linked chloroalkane. In the absence of drug accumulation, HaloTag active sites remain available to react with the fluorophore. Conversely, when a large fraction of molecules of interest permeate the cell membranes and reach the HaloTag proteins, there will be reduced sites available to react with the fluorophore. Therefore, a decrease in cellular fluorescence signal should be reflective.

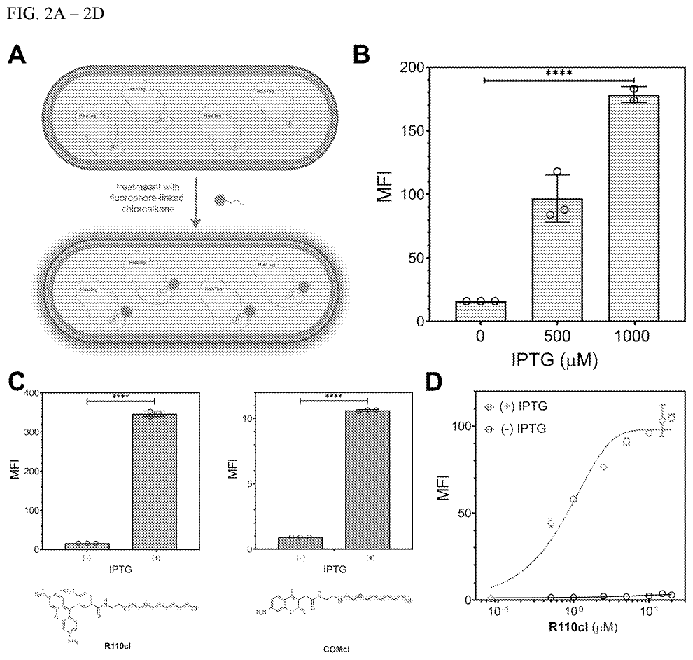

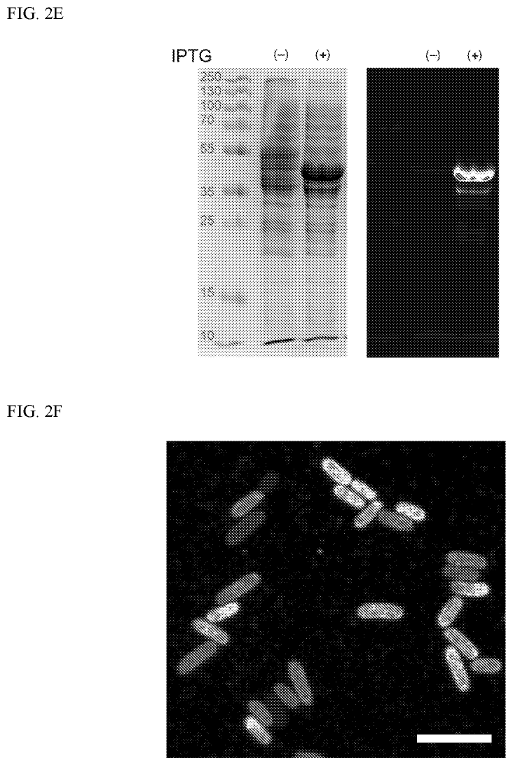

In an embodiment, fluorescence levels were determined in bacterial cells that express HaloTag proteins in the cytosol by encoding the protein on an inducible plasmid. Bacterial cells carrying the HaloTag-expressing plasmid were grown to mid-log, induced with isopropyl-β-D-1-thiogalactopyranoside (IPTG), and incubated with a fluorophore modified chloroalkane. Cytosolic HaloTag promotes the formation of a covalent bond with the fluorophore-linked chloroalkane tail (FIG. 2A). Total labeling levels of bacterial cells is then quantified using flow cytometry. In a first step in the BaCAPA, bacterial cells were treated with Rhodamine 110 modified with chloroalkane (R110cl). A large increase in cellular fluorescence was observed in a dose dependent manner with increasing levels of IPTG (FIG. 2B). These results indicate HaloTag-mediated reporting on the accumulation of R110cl into bacterial cells. Appreciating that the fluorophore itself could be subject to the same barrier elements that are inherent to E. coli, there was also evaluated the labeling of bacterial cells with a smaller fluorophore, namely coumarin (COMcl). Physiochemical properties of these dyes could alter permeation into bacterial cells due to differences in size and charge between the two dyes. While it was clear that coumarin could also covalently modify HaloTag-expressing bacterial cells, the relative fluorescence increase was more modest in cells treated with COMcl (FIG. 2C). Therefore, in illustrative embodiments, R110cl was chosen as the reporter dye.

To empirically establish the dye concentration that optimizes the signal output for the assay, bacterial cells were treated with a range of R110cl concentrations (FIG. 2D). The results showed that 0.5 μM is sufficient to yield fluorescence levels that were nearly 50% of the maximum and by 5 μM the fluorescence signals were reaching saturation levels. For these reasons, all subsequent assays were carried out using 5 μM of R110cl. HaloTag labeling with R110cl can be analyzed from the whole cellular proteome because of the selectivity of this enzymatic reaction. To this end, bacterial cells were treated similar to the previous experiments, subjected to separation on an SDS-PAGE, and the gel was imaged using a fluorescent filter. A clear band corresponds to the molecular weight of HaloTag (FIG. 2E), which strongly indicate that the signals observed from the flow cytometry analysis are representative of HaloTag covalently bound with R110cl inside bacterial cells. Cells were also imaged using confocal microscopy and the labelling pattern was consistent with the projected labeling of HaloTag (FIG. 2F). Together, these results serve to establish the working parameters for BaCAPA.

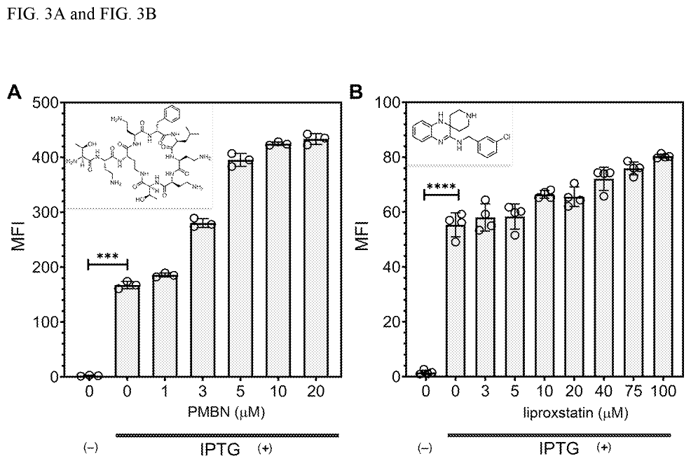

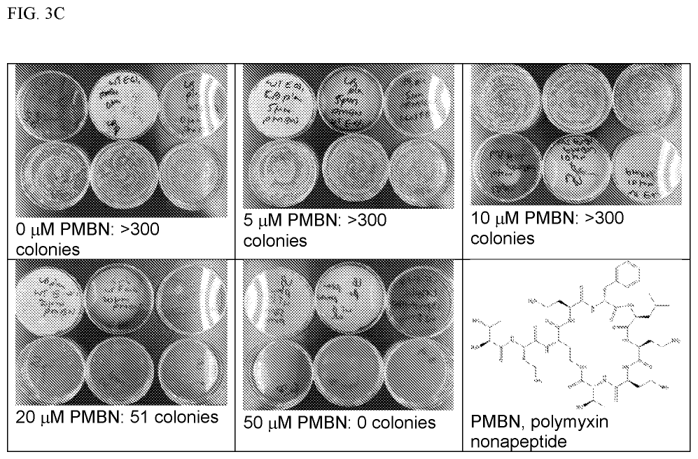

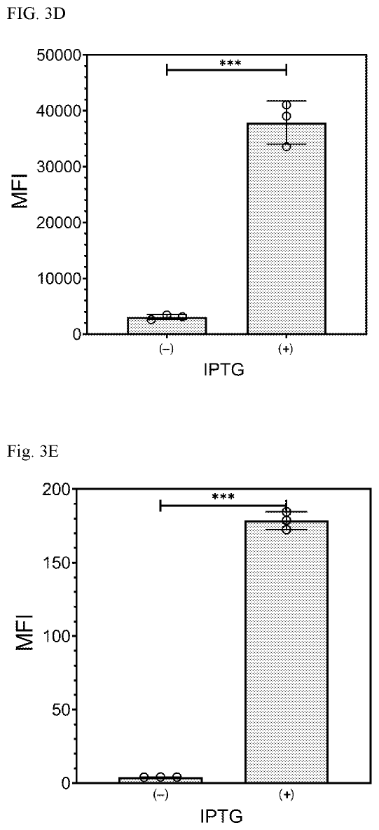

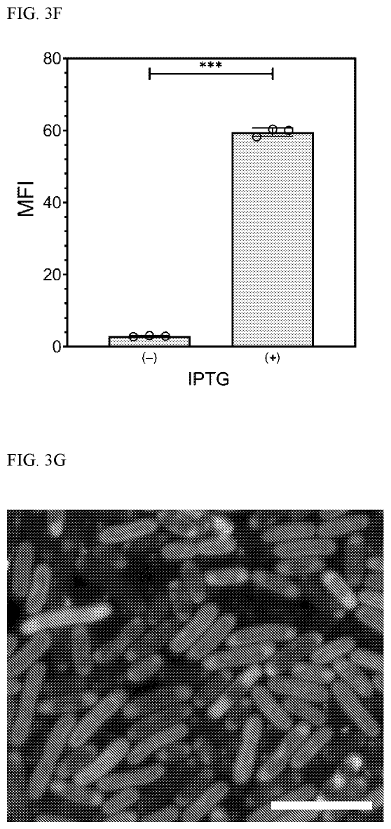

It is well appreciated that the OM is the major barrier to the permeation of small molecules.²⁸ Considerable efforts have been devoted to the discovery of molecules that disrupt the OM barrier as antibiotic adjuvants with the goal of potentiating them and/or circumventing resistance.²⁹⁻³¹ Therefore, these agents hold the promise of synergistically enhancing the permeation of potential antibiotics in a broad and, potentially, impactful way. This class of molecules is best represented by the Gram-negative specific antibiotic polymyxin B. More specifically, a fragment called polymyxin B nonapeptide (PMBN), which lacks the fatty acid tail, has permeabilizing properties at concentrations that are non-toxic to bacterial cells.³² For example, PMBN demonstrated the ability to protect mice against Gram-negative bacteria when dosed with erythromycin.³³ Co-treatment of cells with PMBN and R110cl can introduce a greater level of R110cl to bacterial cells, thus resulting in higher fluorescence levels (FIG. 3A). Indeed, a concentration-dependent increase in cellular fluorescence was observed and the fluorescence levels of cells treated with 10 μM of PMBN was double than that of cells not treated with PMBN. Moreover, no loss of bacterial viability was observed at the concentrations of PMBN tested (FIG. 3C).

A recent screening campaign against a library of 140,000 diverse compounds revealed liproxstatin as a promising new disruptor of E. coli OM.³⁰ Similar to PMBN, an increase in cellular fluorescence was observed when liproxstatin was co-incubated with R110cl (FIG. 3B). These results demonstrate the potential of BaCAPA to be leveraged, more generally, to report on molecules that disrupt the outer membrane of Gram-negative bacteria. In an effort to test the assay compatibility with high-throughput screening platforms, cellular fluorescence levels could be measured via a plate reader rather than flow cytometer: results showed that the assay readily reports on HaloTag-mediated fluorescence (FIG. 3D). While there was a decrease in signal-to-noise, the signal strength in the presence of IPTG was well above that in the absence of IPTG.

In various embodiments, the BaCAPA process can be implemented in a smooth E. coli strain (ATCC 25922) that contains the complete set of lipopolysaccharides on the surface. In contrast to K-12 rough bacteria, smooth E. coli strains can potentially have altered permeability. It has been demonstrated that the surface of rough E. coli has higher accessibility to molecules than smooth E. coli, presumably due to the steric blockade provided by O-antigens.³⁴ Similarly, it is possible that the O-antigens can contribute to the overall permeation profile of small molecules considering that LPS chains have been proposed to occlude porin channels via/by steric shielding.³⁵ Results confirm the feasibility of analyzing BaCAPA in smooth WT E. coli in a similar manner to that of the K-12 E. coli: when induced and non-induced WT E. coli cells were treated with R110cl, there was observed a HaloTag dependent increase in fluorescence; therefore, these results confirmed that the permeability assay can be readily implemented in a WT strain (FIG. 3E). Additionally, we posed that it may be possible to take advantage of directed localization tags to specifically probe the accumulation of molecules within individual compartments in E. coli. To test this, an N-terminus fusion of a DsbA signal peptide was added to HaloTag to transport it to the periplasmic space.³⁶ While we observed minimal fluorescence signals in E. coli BL21(DE3), high fluorescence levels were observed in Lemo21 (DE3) (FIG. 3F). Expression in Lemo21 cells likely afforded a more controlled expression level that may have favorably modulated the folding of HaloTag. Additionally, confocal microscopy established that the DsbA-HaloTag labeling pattern is more consistent with the expected localization within the periplasmic space compared to that of the cytoplasmic HaloTag (FIG. 3G).

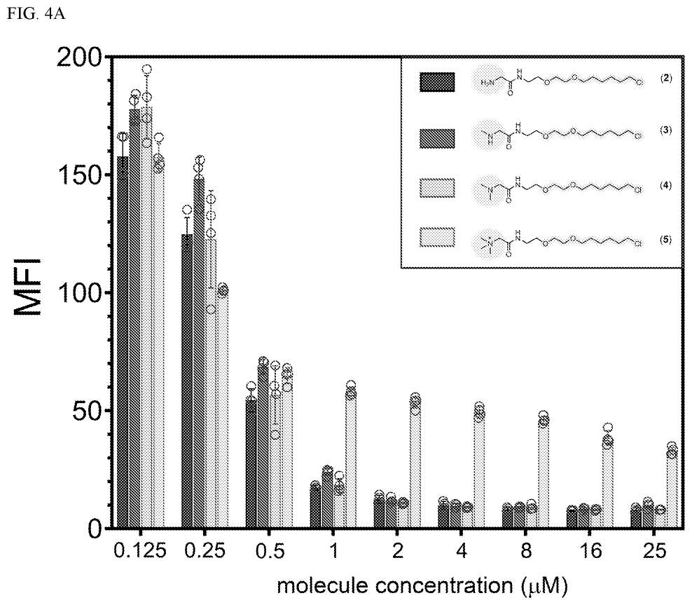

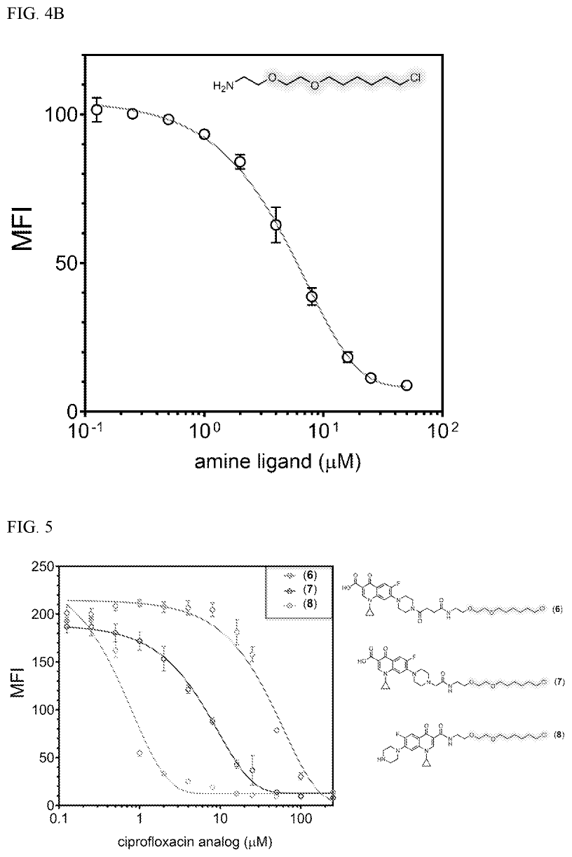

In some embodiments, the BaCAPA process can report on apparent accumulation of small molecules past the cellular envelope. In these embodiments, (non-fluorescent) small molecules modified with the chloroalkane are incubated with the bacterial cells expressing HaloTag first (pulse step), which is then followed by a treatment step with the dye R110cl (chase step). The initial test was performed with a simple amino-ligand (1). A concentration scan of the small molecule revealed that nearly background fluorescence levels were observed at 12 μM with an EC50˜ 5 μM (FIG. 4B). These results demonstrate that BaCAPA reports on the apparent accumulation of small molecules into bacterial cells.

Chemical structure of the chase molecule could potentially impact its permeability into the bacterial cells. In some embodiments, the BaCAPA process was leveraged to evaluate the impact of methylation of a primary amine on relative accumulation in E. coli (FIG. 4A). The inclusion of a primary amine can result in considerably high levels of accumulation in E. coli relative to any other methylation state across a series of molecules (although there were some deviations observed).¹⁹ In the present disclosure, however, data revealed that within the series of tested molecules, there was minimal difference in relative accumulation across the primary, secondary, or tertiary amino group (compounds 2-4) (FIG. 4A). There was, however, an observed and statistically significant lowered accumulation level with the quaternary ammonium derivative (5). Several factors could potentially explain the observations. First, the mode of permeation can be an important molecular determinant towards the accumulation into the bacterial cells and its subsequent potential efflux. Therefore, the 2-4 series could be entering through a different route than prior molecules previously investigated. Alternatively, the illustrative series chosen here has the potential for an internal hydrogen bond,³⁷ of which may alter the desolvation energetics, ultimately impacting permeation profiles across low dielectric environments.

To expand the application of BaCAPA for studying the permeability in E. coli, a group of molecules were synthesized based on the antibiotic ciprofloxacin decorated with that chloroalkane tag. After the discovery of quinolones as potent inhibitors of DNA replication in bacteria, this molecular scaffold was vastly explored in search of more potent analogs, which yielded clinically important agents like norfloxacin, sparfloxacin, levofloxacin, moxifloxacin, gatifloxacin.³⁸ A large fraction of ciprofloxacin analogs involve derivatives to the piperazine moiety, which has shown to be tolerant of functional group manipulations. Therefore, two analogs were built from modifications of the secondary amine for installation of the chloroalkane linker. In one analog, the secondary amine was converted to an amide (6) and, in another, a modification was made with the goal of retaining an ionizable amino group (7). A third molecule was synthesized in which the chloroalkane was, instead, coupled onto the carboxylic acid on the parent molecule (8). The free carboxylic acid is known to be crucial for the antimicrobial activity, and, therefore, we did not expect that this molecule would have biological activity.³⁹ Nonetheless, it provided an opportunity to assess how the permeation could be modulated by making a derivative that should have a net positive charge. Results showed that retention of an ionizable amine group in (7) resulted in improved apparent permeation (EC50=6.5 μM) relative to the similarly structured analog with an amide (6), which had an EC50=42.2 μM (FIG. 5). These two derivatives had the same MIC value of 0.5 μg/mL. These results highlight the deviation that can arise from using MIC values as a proxy for relative permeation of molecules into Gram-negative bacteria. Installment of the chloroalkane ligand in (8) led a significant reduction in EC50 (0.5 μM); nonetheless, the MIC value increased 8-fold to 4 μg/mL.

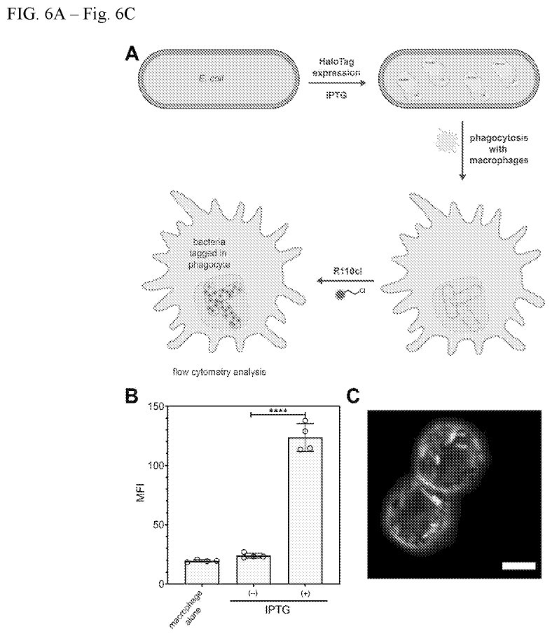

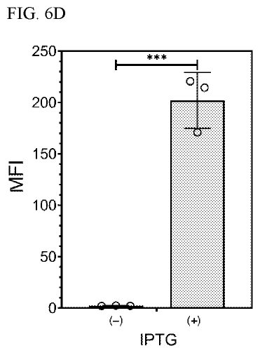

In further embodiments, the BaCAPA process is useful for probing accumulation of small molecules into bacteria residing inside host mammalian cells (FIG. 6A). Given the CAPA previously showed low background labeling of mammalian cells in the absence of HaloTag expression, the same should be expected when those cells are infected with bacteria.^{23, 40-42} The principal reason to establish the feasibility of BaCAPA inside mammalian cells is that a large fraction of serious human pathogens (e.g., Mycobacterium tuberculosis, Salmonella enterica, Chlamydia trachomatis, and Neisseria gonorrhea) can survive (sometimes exclusively) inside host cells.⁴³ Moreover, extracellular bacteria can also temporarily reside inside host cells to avoid the action of antibiotics and to promote tissue dissemination.⁴⁴⁻⁴⁵ The mammalian plasma membrane can compound the challenges in drug accumulation by introducing an additional barrier. Recent advances in the measurement of accumulation of drugs into bacterial cells have not been extended to intracellular bacteria in mammalian cells. The process can result in specific labeling of bacterial cells inside macrophages: prior to exposure to macrophages, HaloTag protein expression was induced with IPTG and after phagocytosis of bacterial cells mammalian cells were treated with R110cl (FIG. 6A). Results revealed that there was a large increase in fluorescence levels when macrophages were incubated with HaloTag expressing bacterial cells (FIG. 6B). The absence of HaloTag induction and macrophages alone showed background levels of fluorescence. The same bacterial cells were also subjected to treatment with R110cl (with no macrophage incubation) and were found to have high levels of fluorescence when induced with IPTG (FIG. 6D). Confocal microscopy confirmed that cellular fluorescence was exclusively localized to bacteria cells (FIG. 6C). These results establish that BaCAPA can surprisingly achieve an unprecedented level of analysis in drug accumulation into bacteria residing inside mammalian cells.

The BaCAPA process readily reports on the accumulation of small molecules in F. coli via irreversible covalent bond formation with HaloTag. Fluorescence signals with a rhodamine-based dye proved to be superior to coumarin, thus resulting in sensitive measurements. BaCAPA can also be adopted to establish the disruption of the outer membrane of E. coli as demonstrated by PMBN and liproxstatin. In a library of small molecules containing an amino group with varying levels of methyl modifications, the methylation level was mostly independent of the apparent accumulation levels. There was synthesized a group of ciprofloxacin analogs and observed that there was a wide range of apparent accumulation based on the structural modifications. Most significantly, there was a prominent discord between apparent accumulation levels and the MIC values of these modified ciprofloxacin. These results highlight the potential challenges in using MIC values as a proxy for drug accumulation, which can confound assignments for structure-activity relationships. Bacteria-expressing HaloTag can specifically labeled inside macrophages in a manner that is compatible with BaCAPA. The results establish the BaCAPA process as a robust and readily adoptable fluorescence-based reporter for the accumulation of molecules into bacterial cells.

### Bacterial Azide Permeability Assay, BAPA

Additional embodiments of the present disclosure are premised upon azides as they are generally considered to be the smallest and least disruptive biorthogonal tags (Jewett J C, Bertozzi C R. Cu-free click cycloaddition reactions in chemical biology. Chem Soc Rev. April 2010; 39(4):1272-9; Agard N J, Prescher J A, Bertozzi C R. A strain-promoted [3+2] azide-alkyne cycloaddition for covalent modification of biomolecules in living systems. J Am Chem Soc. Nov. 24 2004; 126(46):15046-7). Thus, in embodiments, the present disclosure provides a process for determining whether a small molecule is a potential antibiotic drug. The process comprises:
(a1) contacting a bacteria that expresses a mutant form of bacterial haloalkane dehalogenase with a reagent of the general formula (I) to yield a tagged population of bacteria:

[strained alkyne]-(linker)-(alk-Cl) (I)
wherein
[strained alkyne] is a moiety containing a strained alkyne functional group;
(linker) is selected from a bond, amino, amido, carboxyl, C₁-C₆-ester, C₁-C₆-alkoxy, linear and cyclic boronic esters, 3- to 8-membered heterocycloalkyl (wherein 1 to 3 heteroatoms are selected from N, O, and S), C₃-C₈-cycloalkyl optionally fused to 1 or 2 C₆-C₁₀-aryl, C₆-C₁₀-aryl, 5- to 10-membered heteroaryl (wherein 1 to 4 heteroatoms are selected from N, O, and S), succinimidyl, C₁-C₆-alkyl optionally interrupted by one or more of —C(O)— and —N(H)—, and combinations thereof; and
(alk-Cl) is a straight or branched C₁-C₂₀-chloroalkyl optionally interrupted by 1 to 6 oxygen atoms;
(b1) contacting the tagged population of bacteria with the small molecule that has been linked to an azide moiety to yield a first population of treated bacteria;
(c1) contacting the first population of treated bacteria with a fluorophore that is linked to an azide moiety to yield a second population of treated bacteria;
(d1) detecting a fluorescence signal of the second population of treated bacteria; and
(e1) determining that a low fluorescence signal relative to background fluorescence correlates to a conclusion of high accumulation of the small molecule within the bacteria, and that a high fluorescence signal relative to background fluorescence correlates to a conclusion of low accumulation of the small molecule within the bacteria.

In some embodiments, the fluorophore is a rhodamine dye or coumarin. In an exemplary embodiment, the fluorophore is a rhodamine dye, such as Rhodamine 110.

In various embodiments, the [strained alkyne] comprises a C₇-C₁₄-cycloalkyne moiety. Various strained alkynes are known or are commercially available. The particular choice of strained alkyne is not critical, as the strained alkyne is chosen for its readiness to react with an azide in cycloaddition reaction. Thus, in an illustrative embodiment, [strained alkyne] is dibenzocyclooctyne (DBCO).

In additional embodiments, alk-Cl is a C₁₀-C₁₅-chloroalkyl optionally interrupted by 1 to 3 oxygen atoms. These include, for example, the exemplary alk-Cl of the formula:

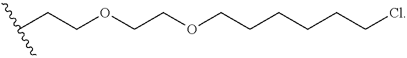 [SMILES: *CCOCCOCCCCCCCl]

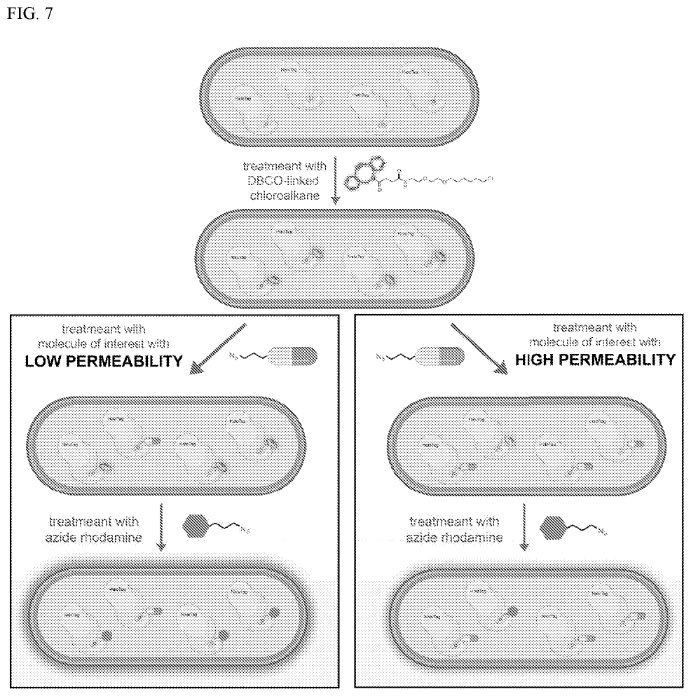

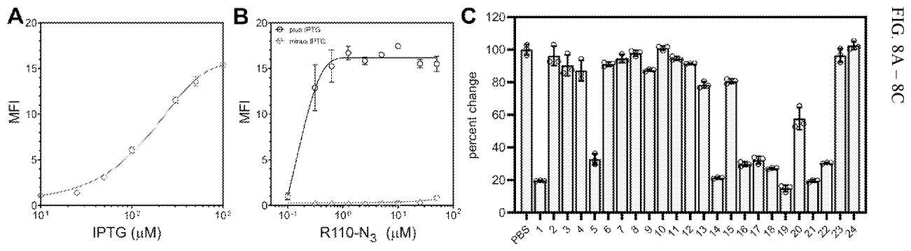

In general, HaloTag-expressing bacterial cells are treated with a compound of formula (I) to anchor a strained-alkyne to the cell. More specifically, in some embodiments, HaloTags are reacted in situ with a dibenzocylcooctyne (DBCO) moiety linked to chloroalkane. Conventional synthetic methodology known to the skilled chemist links the strained alkyne, e.g., DBCO, to the (alk-Cl) moiety through a linker such as a direct bond, and amino group, a succinimidyl group, a C₁-C₅-alkyl optionally interrupted by one or more of —C(O)— and —N(H)—, and combinations thereof. Cyclooctynes are then leveraged to gauge the access of azide-bearing test molecules following in situ strain-promoted azide-alkyne cycloaddition (SPAAC; Spangler B, Dovala D, Sawyer W S, et al. Molecular Probes for the Determination of Subcellular Compound Exposure Profiles in Gram-Negative Bacteria. ACS Infect Dis. Sep. 14 2018; 4(9):1355-1367; Spangler B, Yang S, Baxter Rath C M, Reck F, Feng B Y. A Unified Framework for the Incorporation of Bioorthogonal Compound Exposure Probes within Biological Compartments. ACS Chem Biol. Apr. 19 2019; 14(4):725-734). The general workflow of Bacterial Azide Permeability Assay (BAPA) mirrors that of BaCAPA described herein (FIG. 7).

In the process described herein, the small molecule is a potential antibiotic drug in which a functional group is utilized to tether the small molecule to an azide moiety. Examples of azide reagents useful for this purpose include those in Table 1:

TABLE 1
Examples of Azide Reagents for tagging Small Molecules with Azide

| | |
|---|---|
| 1 | 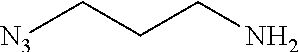 |
| 2 | 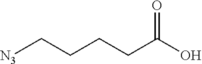 |
| 3 | 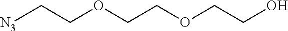 [SMILES: [N-]=[N+]=NCCOCCOCCO] |
| 4 | 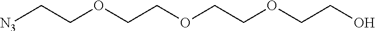 [SMILES: [N-]=[N+]=NCCOCCOCCOCCO] |
| 5 | 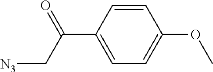 |
| 6 | 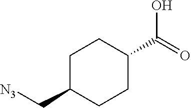 [SMILES: [N-]=[N+]=NC[C@H]1CC[C@H](C(=O)O)CC1] |
| 7 | 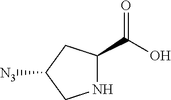 [SMILES: [N-]=[N+]=N[C@H]1CN[C@H](C(=O)O)C1] |
| 8 | 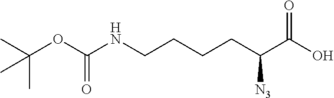 [SMILES: CC(C)(C)OC(=O)NCCCC[C@H](N=[N+]=[N-])C(=O)O] |
| 9 | 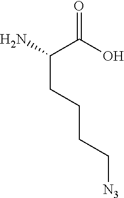 [SMILES: [N-]=[N+]=NCCCC[C@H](N)C(=O)O] |
| 10 | 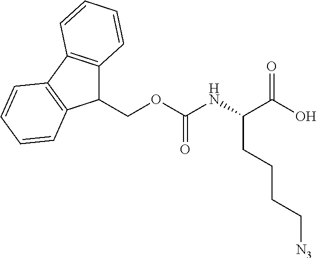 [SMILES: [N-]=[N+]=NCCCC[C@H](NC(=O)OCC1c2ccccc2-c2ccccc21)C(=O)O] |
| 11 | 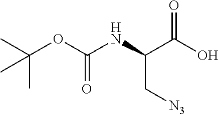 [SMILES: CC(C)(C)OC(=O)N[C@H](CN=[N+]=[N-])C(=O)O] |
| 12 | 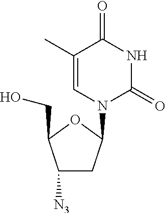 [SMILES: Cc1cn([C@H]2C[C@H](N=[N+]=[N-])[C@@H](CO)O2)c(=O)[nH]c1=O] |
| 13 | 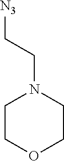 [SMILES: [N-]=[N+]=NCCN1CCOCC1] |
| 14 | 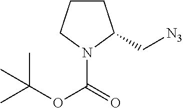 [SMILES: CC(C)(C)OC(=O)N1CCC[C@@H]1CN=[N+]=[N-]] |
| 15 | 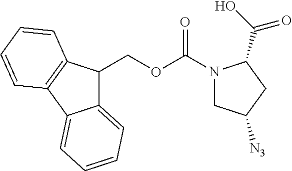 [SMILES: [N-]=[N+]=N[C@H]1C[C@@H](C(=O)O)N(C(=O)OCC2c3ccccc3-c3ccccc32)C1] |
| 16 | 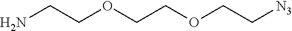 [SMILES: [N-]=[N+]=NCCOCCOCCN] |
| 17 | 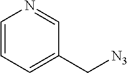 |
| 18 | 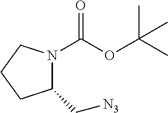 [SMILES: CC(C)(C)OC(=O)N1CCC[C@H]1CN=[N+]=[N-]] |
| 19 | 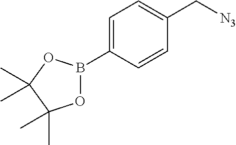 [SMILES: CC1(C)OB(c2ccc(CN=[N+]=[N-])cc2)OC1(C)C] |
| 20 | 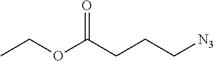 |
| 21 | 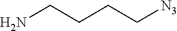 |
| 22 | 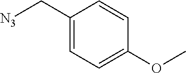 |
| 23 | 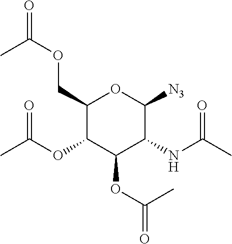 [SMILES: CC(=O)N[C@@H]1[C@@H](OC(C)=O)[C@H](OC(C)=O)[C@@H](COC(C)=O)O[C@H]1N=[N+]=[N-]] |
| 24 | 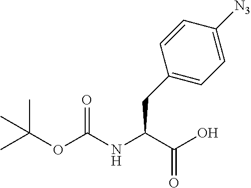 [SMILES: CC(C)(C)OC(=O)N[C@@H](Cc1ccc(N=[N+]=[N-])cc1)C(=O)O] |
| 25 | 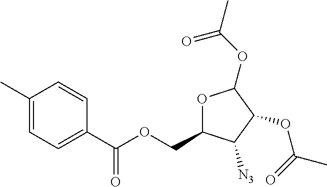 [SMILES: CC(=O)OC1O[C@H](COC(=O)c2ccc(C)cc2)[C@@H](N=[N+]=[N-])[C@H]1OC(C)=O] |

Straight forward organic chemistry informs the choice of small molecule and azide reagent to install the azide moiety onto the small molecule. Illustrative and non-limiting synthetic methodologies for this purpose, for instance in combination with the illustrative azide reagents in Table 1, are shown in Scheme 1 below.

### Scheme 1. Examples of Reactions Between Small Molecule Antibiotic Candidates and Azide Reagents^{a}

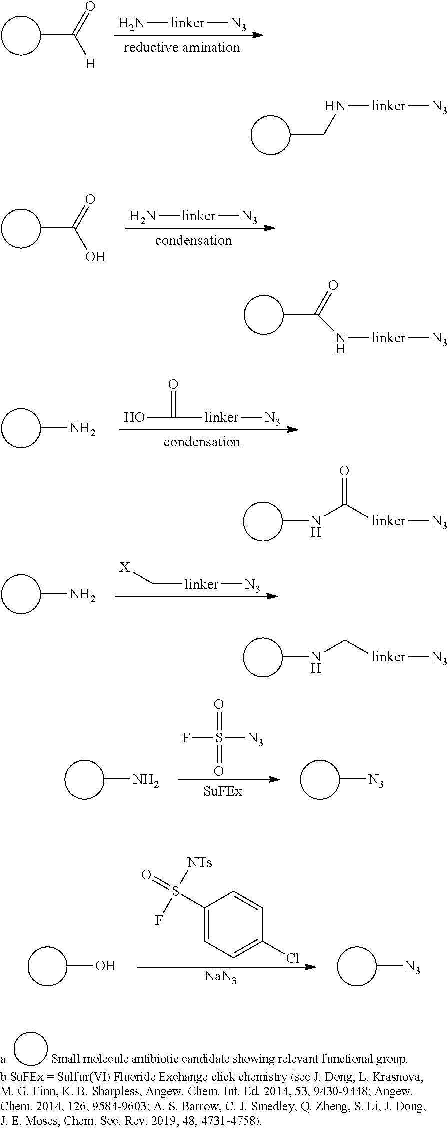

In additional embodiments optionally in combination with any other embodiment described herein, the bacteria in any of the processes is a gram-negative bacteria. For example, in an embodiment, the bacteria is E. coli.

The processes described herein comprise a determination that correlates low fluorescence signal relative to background fluorescence to a conclusion of high accumulation of the small molecule within the bacteria. In contrast, a high fluorescence signal relative to background fluorescence correlates to a conclusion of low accumulation of the small molecule within the bacteria. In some embodiments, the determination is qualitative, such as in an initial step of assaying candidate antibiotic compounds, in that measured fluorescence is below an arbitrary cutoff, such as 50% of total intensity, to support a determination that a small molecule is a potential antibiotic drug.

In other embodiments, such as in one or more subsequent and optionally iterative steps, the determination is semi-quantitative or quantitative. Thus, in these embodiments, a low fluorescence signal is between 0% to 50%, 0% to 40%, 0% to 30%, 0% to 20%, 0% to 10%, or 0% to 5% above background fluorescence (arbitrary intensity units). In contrast, a high fluorescence signal is between 50% to 100%, 60% to 100%, 70% to 100%, 80% to 100%, 90% to 100%, or 95% to 100% above background fluorescence (arbitrary intensity units).

In still further embodiments, the steps in a process described herein are adapted to a high throughput screen on a library of candidate antibiotic compounds. An initial qualitative determination as described above is useful to apply a threshold cutoff to reduce the number of compounds in the library to a subsequent library of fewer compounds that are then subjected to semi-quantitative or quantitative determination for finer assessment of potential antibiotic potential.

Numbered references in the Detailed Description are as follows:
1. Boucher, H. W.; Talbot, G. H.; Bradley, J. S.; Edwards, J. E.; Gilbert, D.; Rice, L. B.; Scheld, M.; Spellberg, B.; Bartlett, J., Bad bugs, no drugs: no ESKAPE! An update from the Infectious Diseases Society of America. Clin Infect Dis 2009, 48(1), 1-12.
2. Payne, D. J.; Gwynn, M. N.; Holmes, D. J.; Pompliano, D. L., Drugs for bad bugs: confronting the challenges of antibacterial discovery. Nat Rev Drug Discov 2007, 6 (1), 29-40.
3. Tommasi, R.; Brown, D. G.; Walkup, G. K.; Manchester, J. I.; Miller, A. A., ESKAPEing the labyrinth of antibacterial discovery. Nat Rev Drug Discov 2015, 14 (8), 529-42.
4. Culp, E. J.; Waglechner, N.; Wang, W.; Fiebig-Comyn, A. A.; Hsu, Y. P.; Koteva, K.; Sychantha, D.; Coombes, B. K.; Van Nieuwenhze, M. S.; Brun, Y. V.; Wright, G. D., Evolution-guided discovery of antibiotics that inhibit peptidoglycan remodelling. Nature 2020, 578(7796), 582-587.
5. Durand-Reville, T. F.; Miller, A. A.; O'Donnell, J. P.; Wu, X.; Sylvester, M. A.; Guler, S.; Iyer, R.; Shapiro, A. B.; Carter, N. M.; Velez-Vega, C.; Moussa, S. H.; McLeod, S. M.; Chen, A.; Tanudra, A. M.; Zhang, J.; Comita-Prevoir, J.; Romero, J. A.; Huynh, H.; Ferguson, A. D.; Horanyi, P. S.; Mayclin, S. J.; Heine, H. S.; Drusano, G. L.; Cummings, J. E.; Slayden, R. A.; Tommasi, R. A., Rational design of a new antibiotic class for drug-resistant infections. Nature 2021, 597(7878), 698-702.
6. Imai, Y.; Meyer, K. J.; Iinishi, A.; Favre-Godal, Q.; Green, R.; Manuse, S.; Caboni, M.; Mori, M.; Niles, S.; Ghiglieri, M.; Honrao, C.; Ma, X.; Guo, J. J.; Makriyannis, A.; Linares-Otoya, L.; Bohringer, N.; Wuisan, Z. G.; Kaur, H.; Wu, R.; Mateus, A.; Typas, A.; Savitski, M. M.; Espinoza, J. L.; O'Rourke, A.; Nelson, K. E.; Hiller, S.; Noinaj, N.; Schaberle, T. F.; D'Onofrio, A.; Lewis, K., A new antibiotic selectively kills Gram-negative pathogens. Nature 2019, 576(7787), 459-464.
7. Le, P.; Kunold, E.; Macsics, R.; Rox, K.; Jennings, M. C.; Ugur, I.; Reinecke, M.; Chaves-Moreno, D.; Hackl, M. W.; Fetzer, C.; Mandl, F. A. M.; Lehmann, J.; Korotkov, V. S.; Hacker, S. M.; Kuster, B.; Antes, I.; Pieper, D. H.; Rohde, M.; Wuest, W. M.; Medina, E.; Sieber, S. A., Repurposing human kinase inhibitors to create an antibiotic active against drug-resistant Staphylococcus aureus, persisters and biofilms. Nat Chem 2020, 12(2), 145-158.
8. Leimer, N.; Wu, X.; Imai, Y.; Morrissette, M.; Pitt, N.; Favre-Godal, Q.; Iinishi, A.; Jain, S.; Caboni, M.; Leus, I. V.; Bonifay, V.; Niles, S.; Bargabos, R.; Ghiglieri, M.; Corsetti, R.; Krumpoch, M.; Fox, G.; Son, S.; Klepacki, D.; Polikanov, Y. S.; Freliech, C. A.; Mccarthy, J. E.; Edmondson, D. G.; Norris, S. J.; D'Onofrio, A.; Hu, L. T.; Zgurskaya, H. I.; Lewis, K., A selective antibiotic for Lyme disease. Cell 2021, 184(21), 5405-5418 e16.
9. Ling, L. L.; Schneider, T.; Peoples, A. J.; Spoering, A. L.; Engels, I.; Conlon, B. P.; Mueller, A.; Schaberle, T. F.; Hughes, D. E.; Epstein, S.; Jones, M.; Lazarides, L.; Steadman, V. A.; Cohen, D. R.; Felix, C. R.; Fetterman, K. A.; Millett, W. P.; Nitti, A. G.; Zullo, A. M.; Chen, C.; Lewis, K., A new antibiotic kills pathogens without detectable resistance. Nature 2015, 517(7535), 455-9.
10. Zipperer, A.; Konnerth, M. C.; Laux, C.; Berscheid, A.; Janek, D.; Weidenmaier, C.; Burian, M.; Schilling, N. A.; Slavetinsky, C.; Marschal, M.; Willmann, M.; Kalbacher, H.; Schittek, B.; Brotz-Oesterhelt, H.; Grond, S.; Peschel, A.; Krismer, B., Human commensals producing a novel antibiotic impair pathogen colonization. Nature 2016, 535(7613), 511-6.
11. Sun, J.; Rutherford, S. T.; Silhavy, T. J.; Huang, K. C., Physical properties of the bacterial outer membrane. Nat Rev Microbiol 2021.
12. Vergalli, J.; Bodrenko, I. V.; Masi, M.; Moynie, L.; Acosta-Gutierrez, S.; Naismith, J. H.; Davin-Regli, A.; Ceccarelli, M.; van den Berg, B.; Winterhalter, M.; Pages, J. M., Porins and small-molecule translocation across the outer membrane of Gram-negative bacteria. Nat Rev Microbiol 2020, 18(3), 164-176.
13. Pages, J. M.; James, C. E.; Winterhalter, M., The porin and the permeating antibiotic: a selective diffusion barrier in Gram-negative bacteria. Nat Rev Microbiol 2008, 6 (12), 893-903.
14. Du, D.; Wang-Kan, X.; Neuberger, A.; van Veen, H. W.; Pos, K. M.; Piddock, L. J. V.; Luisi, B. F., Multidrug efflux pumps: structure, function and regulation. Nat Rev Microbiol 2018, 16(9), 523-539.
15. Nikaido, H.; Pages, J. M., Broad-specificity efflux pumps and their role in multidrug resistance of Gram-negative bacteria. FEMS Microbiol Rev 2012, 36(2), 340-63.
16. Masi, M.; Refregiers, M.; Pos, K. M.; Pages, J. M., Mechanisms of envelope permeability and antibiotic influx and efflux in Gram-negative bacteria. Nat Microbiol 2017, 2, 17001.
17. Nikaido, H., Prevention of drug access to bacterial targets: permeability barriers and active efflux. Science 1994, 264(5157), 382-8.
18. Six, D. A.; Krucker, T.; Leeds, J. A., Advances and challenges in bacterial compound accumulation assays for drug discovery. Curr Opin Chem Biol 2018, 44, 9-15.
19. Richter, M. F.; Drown, B. S.; Riley, A. P.; Garcia, A.; Shirai, T.; Svec, R. L.; Hergenrother, P. J., Predictive compound accumulation rules yield a broad-spectrum antibiotic. Nature 2017, 545(7654), 299-304.
20. Davis, T. D.; Gerry, C. J.; Tan, D. S., General platform for systematic quantitative evaluation of small-molecule permeability in bacteria. ACS Chem Biol 2014, 9(11), 2535-44.
21. Spangler, B.; Yang, S.; Baxter Rath, C. M.; Reck, F.; Feng, B. Y., A Unified Framework for the Incorporation of Bioorthogonal Compound Exposure Probes within Biological Compartments. ACS Chem Biol 2019, 14(4), 725-734.
22. Spangler, B.; Dovala, D.; Sawyer, W. S.; Thompson, K. V.; Six, D. A.; Reck, F.; Feng, B. Y., Molecular Probes for the Determination of Subcellular Compound Exposure Profiles in Gram-Negative Bacteria. ACS Infect Dis 2018, 4(9), 1355-1367.
23. Peraro, L.; Deprey, K. L.; Moser, M. K.; Zou, Z.; Ball, H. L.; Levine, B.; Kritzer, J. A., Cell Penetration Profiling Using the Chloroalkane Penetration Assay. J Am Chem Soc 2018, 140(36), 11360-11369.
24. Los, G. V.; Encell, L. P.; McDougall, M. G.; Hartzell, D. D.; Karassina, N.; Zimprich, C.; Wood, M. G.; Learish, R.; Ohana, R. F.; Urh, M.; Simpson, D.; Mendez, J.; Zimmerman, K.; Otto, P.; Vidugiris, G.; Zhu, J.; Darzins, A.; Klaubert, D. H.; Bulleit, R. F.; Wood, K. V., HaloTag: a novel protein labeling technology for cell imaging and protein analysis. ACS Chem Biol 2008, 3(6), 373-82.
25. Ke, N.; Landgraf, D.; Paulsson, J.; Berkmen, M., Visualization of Periplasmic and Cytoplasmic Proteins with a Self-Labeling Protein Tag. J Bacteriol 2016, 198(7), 1035-43
26. Clark, S. A.; Singh, V.; Vega Mendoza, D.; Margolin, W.; Kool, E. T., Light-Up “Channel Dyes” for Haloalkane-Based Protein Labeling in Vitro and in Bacterial Cells. Bioconjug Chem 2016, 27(12), 2839-2843.
27. Yang, Z.; Weisshaar, J. C., HaloTag Assay Suggests Common Mechanism of E. coli Membrane Permeabilization Induced by Cationic Peptides. ACS Chem Biol 2018, 13 (8), 2161-2169.
28. Sun, J.; Rutherford, S. T.; Silhavy, T. J.; Huang, K. C., Physical properties of the bacterial outer membrane. Nat Rev Microbiol 2022, 20(4), 236-248.
29. Stokes, J. M.; MacNair, C. R.; Ilyas, B.; French, S.; Cote, J. P.; Bouwman, C.; Farha, M. A.; Sieron, A. O.; Whitfield, C.; Coombes, B. K.; Brown, E. D., Pentamidine sensitizes Gram-negative pathogens to antibiotics and overcomes acquired colistin resistance. Nat Microbiol 2017, 2, 17028.
30. Klobucar, K.; Cote, J. P.; French, S.; Borrillo, L.; Guo, A. B. Y.; Serrano-Wu, M. H.; Lee, K. K.; Hubbard, B.; Johnson, J. W.; Gaulin, J. L.; Magolan, J.; Hung, D. T.; Brown, E. D., Chemical Screen for Vancomycin Antagonism Uncovers Probes of the Gram-Negative Outer Membrane. ACS Chem Biol 2021, 16(5), 929-942.
31. Vaara, M., Agents that increase the permeability of the outer membrane. Microbiol Rev 1992, 56(3), 395-411.
32. French, S.; Farha, M.; Ellis, M. J.; Sameer, Z.; Cote, J. P.; Cotroneo, N.; Lister, T.; Rubio, A.; Brown, E. D., Potentiation of Antibiotics against Gram-Negative Bacteria by Polymyxin B Analogue SPR741 from Unique Perturbation of the Outer Membrane. ACS Infect Dis 2020, 6(6), 1405-1412.
33. Ofek, I.; Cohen, S.; Rahmani, R.; Kabha, K.; Tamarkin, D.; Herzig, Y.; Rubinstein, E., Antibacterial synergism of polymyxin B nonapeptide and hydrophobic antibiotics in experimental gram-negative infections in mice. Antimicrob Agents Chemother 1994, 38(2), 374-7.
34. Feigman, M. S.; Kim, S.; Pidgeon, S. E.; Yu, Y.; Ongwae, G. M.; Patel, D. S.; Regen, S.; Im, W.; Pires, M. M., Synthetic Immunotherapeutics against Gram-negative Pathogens. Cell Chem Biol 2018, 25(10), 1185-1194 e5.
35. Lee, J.; Patel, D. S.; Kucharska, I.; Tamm, L. K.; Im, W., Refinement of OprH-LPS Interactions by Molecular Simulations. Biophys J 2017, 112(2), 346-355.
36. Schierle, C. F.; Berkmen, M.; Huber, D.; Kumamoto, C.; Boyd, D.; Beckwith, J., The DsbA signal sequence directs efficient, cotranslational export of passenger proteins to the Escherichia coli periplasm via the signal recognition particle pathway. J Bacteriol 2003, 185(19), 5706-13.
37. Giordanetto, F.; Tyrchan, C.; Ulander, J., Intramolecular Hydrogen Bond Expectations in Medicinal Chemistry. ACS Med Chem Lett 2017, 8(2), 139-142.
38. Appelbaum, P. C.; Hunter, P. A., The fluoroquinolone antibacterials: past, present and future perspectives. Int J Antimicrob Agents 2000, 16(1), 5-15.
39. Chu, D. T.; Fernandes, P. B., Structure-activity relationships of the fluoroquinolones. Antimicrob Agents Chemother 1989, 33(2), 131-5.
40. Deprey, K.; Batistatou, N.; Debets, M. F.; Godfrey, J.; VanderWall, K. B.; Miles, R. R.; Shehaj, L.; Guo, J.; Andreucci, A.; Kandasamy, P.; Lu, G.; Shimizu, M.; Vargeese, C.; Kritzer, J. A., Quantitative Measurement of Cytosolic and Nuclear Penetration of Oligonucleotide Therapeutics. ACS Chem Biol 2022, 17(2), 348-360.
41. Deprey, K.; Kritzer, J. A., Quantitative measurement of cytosolic penetration using the chloroalkane penetration assay. Methods Enzymol 2020, 641, 277-309.
42. Gray, J. P.; Uddin, M. N.; Chaudhari, R.; Sutton, M. N.; Yang, H.; Rask, P.; Locke, H.; Engel, B. J.; Batistatou, N.; Wang, J.; Grindel, B. J.; Bhattacharya, P.; Gammon, S. T.; Zhang, S.; Piwnica-Worms, D.; Kritzer, J. A.; Lu, Z.; Bast, R. C., Jr.; Millward, S. W., Directed evolution of cyclic peptides for inhibition of autophagy. Chem Sci 2021, 12(10), 3526-3543.
43. Ray, K.; Marteyn, B.; Sansonetti, P. J.; Tang, C. M., Life on the inside: the intracellular lifestyle of cytosolic bacteria. Nat Rev Microbiol 2009, 7(5), 333-40.
44. Liu, Y.; Jia, Y.; Yang, K.; Wang, Z., Heterogeneous Strategies to Eliminate Intracellular Bacterial Pathogens. Front Microbiol 2020, 11, 563.
45. Kamaruzzaman, N. F.; Kendall, S.; Good, L., Targeting the hard to reach: challenges and novel strategies in the treatment of intracellular bacterial infections. Br J Pharmacol 2017, 174(14), 2225-2236.

### EXAMPLES

### General

All reagent grade reagents were purchased from commercial vendors and were used without further purification. Anhydrous solvents dichloromethane (DCM), methanol, tetrahydrofuran (THF), and dimethylformamide (DMF) were purchased from Sigma. LB Broth (Miller formulation), Mueller Hinton Broth 2, triethylamine, bromoacetyl bromide, and ciprofloxacin were also purchased from Sigma. Vancomycin hydrochloride, anhydrous betaine, Boc-sarcosine N-hydroxysuccinimide ester, and Boc-glycine N-hydroxysuccinimide ester were all purchased from ChemImpex. N,N-Dimethylglycine and di-isopropyl ethylamine (DIPEA) were both purchased from TCI America. 2-(2-((6-Chlorohexyl)oxy)ethoxy)ethanamine hydrochloride was purchased from AmBeed; this compound is called HaloTag amine ligand hereinafter. HaloTag® Coumarin Ligand (chloroalkane-tagged coumarin) and HaloTag® R110Direct™ Ligand (chloroalkane-tagged rhodamine 110) were both purchased from Promega. All reaction vessels were flame dried prior to use. Reaction progress with ciprofloxacin was monitored by thin-layer chromatography, visualized with UV light. Compounds were purified via reversed phase HPLC using Luna 10 μm C8(2) 100 Å LC Column 250×21.2 mm (Phenomenex) with a Waters 1525 Binary HPLC solvent pump. Purity of compounds was confirmed via reversed phase HPLC using a Luna 5 mm C8(2) 100 Å LC Column 250×4.6 mm (Phenomenex) with a Waters 1525 Binary HPLC solvent pump. UV-visible spectra were collected on Thermo Scientific Genesys-50 spectrophotometer using either transparent plastic or quartz cuvettes. 1H and ¹³C-NMR spectra for all new compounds and intermediates for characterization were acquired on a Varian 600 MHz spectrophotometer. All NMR spectra were analyzed using MestreNova software. Residual solvent signal from CDCl₃, CD₃OD and DMSO-d6 referenced to tetramethylsilane (TMS) were used as reference standards for defining chemical shifts of ¹H or ¹³C spectra of compounds. Chemical shifts are reported in 8 ppm and coupling constants (J) are reported in Hertz [Hz]. Deuterated solvents were used as received from Cambridge Isotopes. Routine mass analysis was performed on Advion Expression® CMS mass spectrometer using standard parameters for intermediates. For the analysis of fragmentation sensitive compounds, low fragmentation, low energy setup was used. The observed molecular weights for compounds were represented as m/z. High resolution electrospray ionization mass spectrometry (HRMS, ESI/MS) analyses were obtained on an Agilent 6545B Q-TOF LC/MS equipped with 1260 Infinity II LC system with auto sampler. Samples were dissolved in either methanol or MeCN and eluted with a MeCN/H₂O solution containing 0.1% formic acid. Fluorescence measurement for the probes was performed on Synergy H1 multimode hybrid microplate reader from BioTek with appropriate setting for fluorophores. The HPLC fractions of the desired purified compounds were first concentrated under reduced pressure using rotary evaporator Hei-CHILL (Heidolph). The final concentrated aqueous solutions were lyophilized to dryness using Labconco Freezone 4.5 L (−84° C.) lyophilizer.

### Synthesis and Characterization of Chloroalkane-Tagged Derivatives

### Synthesis of Glycinamide-HaloTag Derivatives

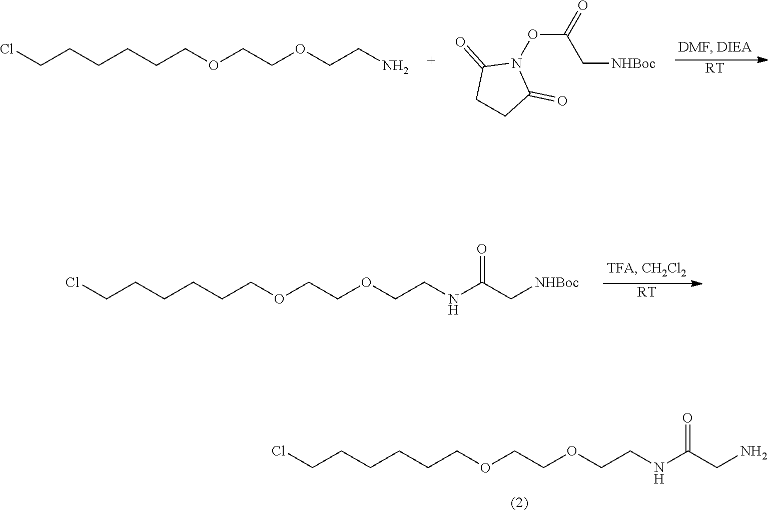 [SMILES: NCCOCCOCCCCCCCl ; CC(C)(C)OC(=O)NCC(=O)ON1C(=O)CCC1=O ; "RT" ; CC(C)(C)OC(=O)NCC(=O)NCCOCCOCCCCCCCl ; "RT" ; NCC(=O)NCCOCCOCCCCCCCl]

Compound (2), HaloTag-glycinamide (R=H): To a solution of N-O'Boc-glycine-NHS ester (55 mg, 0.2 mmol) in DMF (3.0 mL) was first added DIEA (300 μL), followed by HaloTag amine ligand (52.0 mg, 0.2 mmol) at room temperature. The mixture was stirred overnight, diluted with ether (20 mL), washed with dil. HCl (0.1M), water and finally with 10% NaHCO₃ solution. The product was dried over Na₂SO₄, concentrated under reduced pressure, and the leftover product was used as is. The N-^{t}Boc deprotection is performed with 10% TFA in CH₂Cl₂. The mixture was concentrated and subjected to preparative HPLC purification. The fractions showing the desired mass were collected, concentrated under reduced pressure, and finally lyophilized to yield a transparent syrup (39.0 mg, yield: 69%). ¹H-NMR (CDCl₃) δ 1.34 (m, 2H, CH₂), 1.44 (m, 2H, CH₂), 1.57 (m, 2H, CH₂), 1.76 (m, 2H, CH₂), 3.44-3.48. (m, 4H, CH₂), 3.50-3.60 (m, 8H, OCH₂), 3.78 (m, 2H, CH₂), 7.76 (brs, 1H, NH), 8.11 (brs, 2H, NH₂). ¹³C-NMR (CDCl₃) d 25.4, 26.8, 29.3, 32.6, 39.7, 41.2, 45.2, 69.5, 69.8, 70.2, 71.3, 166.7. HRMS: Obs. 309.1970 for [M+H]; Calc. for C₁₄H₃₀ClN₂O₃ 309.1939.

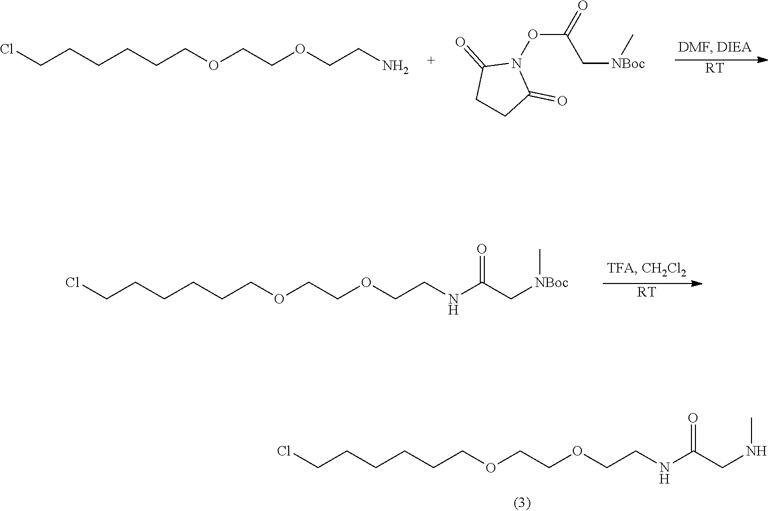 [SMILES: NCCOCCOCCCCCCCl ; CN(CC(=O)ON1C(=O)CCC1=O)C(=O)OC(C)(C)C ; "RT" ; CN(CC(=O)NCCOCCOCCCCCCCl)C(=O)OC(C)(C)C ; "RT" ; CNCC(=O)NCCOCCOCCCCCCCl]

Compound (3), HaloTag-N-methyl glycinamide: Following the foregoing procedure, except for the use of N-′Boc-sarcosine-NHS ester (61.0 mg, 0.21 mmol), afforded a transparent syrup (47.0 mg, yield: 76%). ¹H-NMR (CDCl₃) δ 1.35 (m, 2H, CH₂), 1.44 (m, 2H, CH₂), 1.59 (m, 2H, CH₂), 1.77 (m, 2H, CH₂), 2.80 (brs, 3H, NCH₃), 3.47 (m, 4H, CH₂), 3.53 (t, J=6 Hz 4H, 2×CH₂), 3.54-3.59 (m, 10H, CH₂), 3.85 (brs, 2H, CH₂), 7.53 (brs, 1H, NH), 8.10 (brs, 1H, NH). ¹³C-NMR (CDCl₃) d 25.4, 26.7, 29.3, 32.6, 33.9, 39.8, 45.1, 50.5, 69.3, 69.9, 70.3, 71.4, 165.3. HRMS: Obs. 309.1970 for [M+H]; Calc. for C₁₄H₃₀ClN₂O₃ 309.1939.

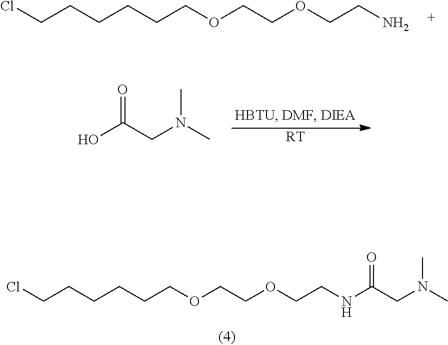

Compound (4), HaloTag-N, N-Dimethyl glycinamide: To a solution of N, N-dimethylglycine (33 mg, 0.32 mmol) in anhydrous DMF (2.0 mL), DIEA (250 μL, 1.43 mol) and HBTU (185 mg, 0.49 mmol) were added and stirred for 10 minutes. Subsequently, HaloTag amine ligand (86 mg, 0.335 mmol) was added to the mixture at room temperature. The mixture was stirred overnight, diluted with ether (25 mL), washed with water and 10% NaHCO₃ solution, dried over Na₂SO₄, and concentrated under reduced pressure. The crude product was purified by preparative HPLC. The fractions showing m/z 309 [M+H] were collected, concentrated under reduced pressure, and finally lyophilized to yield a transparent syrup (71.0 mg, yield: 72%). ¹H-NMR (CDCl₃) δ 1.34 (m, 2H, CH₂), 1.45 (m, 2H, CH₂), 1.58 (m, 2H, CH₂), 1.78 (m, 2H, CH₂), 2.97 (s, 6H, NCH₃), 3.44-3.48 (m, 4H, CH₂), 3.52-3.59 (m, 8H, 4×CH₂, OCH₂), 3.83 (s, 2H, CH₂), 7.99 (brs, 1H, NH). ¹³C-NMR (CDCl₃) d 25.4, 26.7, 29.5, 32.6, 38.7, 45.0, 46.0, 63.16, 70.0, 70.1, 70.3, 71.3, 170.7. HRMS: Obs. 309.1970 for [M+H]; Calc. for C₁₄H₃₀ClN₂O₃ 309.1939.

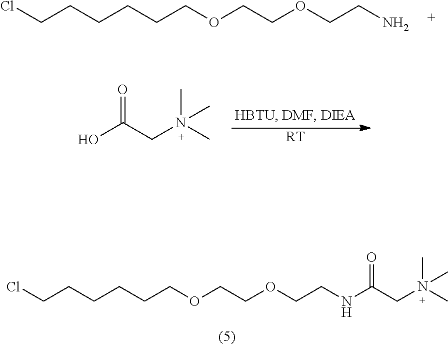

Compound (5), HaloTag-betainamide: To a solution of betaine (35.5 mg, 0.3 mmol) in 1 mL DMF was added DIEA (250 μL, 1.43 mmol) and HBTU (185 mg, 0.49 mmol), and stirred for 10 minutes. Subsequently, HaloTag amine ligand (86 mg, 0.335 mmol) was added to the mixture at room temperature. The mixture was stirred overnight, diluted with ether (25 mL), washed with water and 10% NaHCO₃ solution, dried over Na₂SO₄, and concentrated under reduced pressure. Upon reaction, the mixture was diluted with HPLC solvent (9 mL, 35% ACN: 65% water, with 0.1% TFA) and was directly loaded on preparative HPLC. Using the same gradient used for other compounds, fractions showing m/z 323 [M]⁺ were collected, concentrated under reduced pressure, and finally lyophilized to yield a transparent syrup (57 mg, yield: 59%). ¹H-NMR (CDCl₃) δ 1.35 (m, 2H, CH₂), 1.45 (m, 2H, CH₂), 1.57 (m, 2H, CH₂), 1.76 (m, 2H, CH₂), 3.35 (s, 9H, CH₃), 3.44 (t, 2H, CH₂), 3.47 (m, 2H, CH₂), 3.50-3.60 (m, 8H, OCH₂), 4.28 (s, 2H, CH₂), 8.39 (brs, 1H, NH). ¹³C-NMR (CDCl₃) d 25.4, 26.7, 29.6, 32.6, 39.5, 45.2, 54.7, 65.4, 68.9, 70.0, 70.3, 71.3, 162.8. HRMS: Obs. 323.2176 for [M+H]⁺ Calc. for C₁₅H₃₂ClN₂O₃ 321.2096.

### Synthesis of Compound (6)

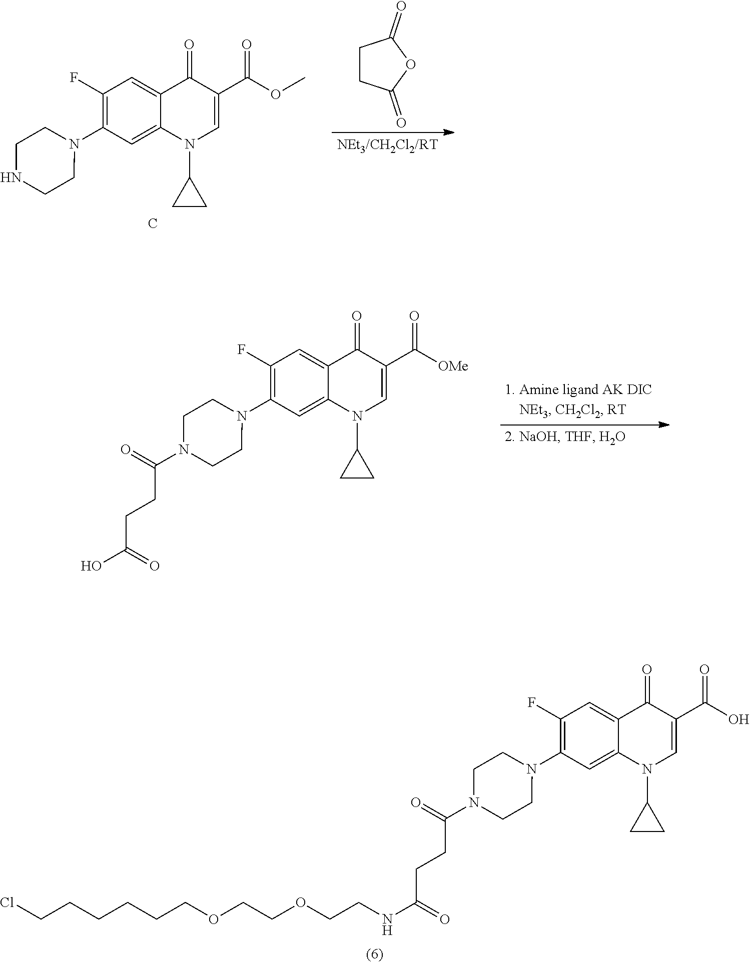

Compound (6). Synthesis of N-succinamide of ciprofloxacin methyl ester: The Ciprofloxacin methyl ester as described below (86 mg, 0.25 mmol) was dissolved in 5 mL of anhydrous CH₂Cl₂ and to this, NEt₃ (100 mg, 1 mmol) was added. To this mixture, succinic anhydride was added as solid (30 mg, 0.3 mmol) and the homogenous mixture stirred at room temperature overnight. TLC analysis revealed that the starting methyl ester was consumed to form a new, non-polar compound. The reaction mixture was diluted with CH₂Cl₂(20 mL), transferred to a separatory funnel, and to this, dil. HCl (5 mL, 0.1M) was added. The aqueous layer was separated, and the organic layer was further washed with water and brine. The organic layer was dried over Na₂SO₄ and concentrated under reduced pressure. Column chromatography purification over silica-gel (hexanes/ethyl acetate, 1:1) afforded a pure white solid (82 mg, yield: 74%). ¹H-NMR: (CDCl₃) δ 1.14 (m, 2H, cyclopropyl-CH₂), 1.32 (m, 2H, cyclopropyl-CH₂), 1.39-1.49 (m, 6H, 3×CH₂), 1.49 (s, 9H, 3×CH₃), 1.58 (dt, J=6 Hz, 128 Hz, 2H, CH₂), 1.74 (dt, J=6, 12 Hz, 2H, CH₂), 3.22 (t, J=6 Hz, 4H, 2×CH₂), 3.43 (m, 1H, CH), 3.45-3.51 (m, 4H, piperidyl 2×CH₂), 3.62 (m, 2H), 3.63-3.67 (m, 4H, 2×CH₂), 7.31 (d, J=6 Hz, 1H, ArH), 8.03 (d, J=12 Hz, 1H, ArH), 8.81 (s, 1H, ArH), 10.1 (brs, 1H, NH). ¹³C-NMR: (CDCl₃) d 8.2, 25.4, 26.8, 28.4, 29.5, 32.6, 34.7, 39.2, 45.1, 50.0, 70.0, 70.2, 70.6, 71.3, 105.0, 111.4, 112.7, 112.8, 122.2, 138.4, 144.8, 146.8, 152.6, 154.6, 165.1, 175.4.

Synthesis of HaloTag amide from N-succinyl ciprofloxacin methyl ester: N-Succinyl-ciprofloxacin methyl ester (45 mg, 0.1 mmol) was dissolved in CH₂Cl₂ containing NEt₃ (101 mg, 1 mmol). To this, DIC (20 μL, 16.1 mg, 0.13 mmol) and HaloTag amine ligand (31 mg, 0.12 mmol) were added and stirred at room temperature overnight. TLC analysis showed that the starting material was completely consumed to form a new compound of a different polarity. The reaction mixture was concentrated and purified by silica-gel column chromatography with CHCl₃/MeOH (98:2) eluent. The pure compound was isolated after concentration and drying (51 mg, yield: 78%). ¹H-NMR: (CDCl₃) δ 1.14 (m, 2H, cyclopropyl-CH₂), 1.32 (m, 2H, cyclopropyl-CH₂), 1.39-1.49 (m, 6H, 3×CH₂), 1.49 (s, 9H, 3×CH₃), 1.58 (dt, J=6 Hz, 128 Hz, 2H, CH₂), 1.74 (dt, J=6, 12 Hz, 2H, CH₂), 3.22 (t, J=6 Hz, 4H, 2×CH₂), 3.43 (m, 1H, CH), 3.45-3.51 (m, 4H, piperidyl 2×CH₂), 3.62 (m, 2H), 3.63-3.67 (m, 4H, 2×CH₂), 7.31 (d, J=6 Hz, 1H, ArH), 8.03 (d, J=12 Hz, 1H, ArH), 8.81 (s, 1H, ArH), 10.1 (brs, 1H, NH). ¹³C-NMR: (CDCl₃) d 8.2, 25.4, 26.8, 28.4, 29.5, 32.6, 34.7, 39.2, 45.1, 50.0, 70.0, 70.2, 70.6, 71.3, 105.0, 111.4, 112.7, 112.8, 122.2, 138.4, 144.8, 146.8, 152.6, 154.6, 165.1, 175.4.

Hydrolysis of the methyl ester: The ciprofloxacin-N-acylated HaloTag amide (33 mg, 0.05 mmmol) was dissolved in THF (2.0 mL) and to this was added 1.0M NaOH (200 μL). The mixture was vigorously stirred at room temperature for 12 hrs. TLC analysis showed that the methyl ester was consumed to form a new polar compound. The reaction mixture was concentrated under reduced pressure and acidified with dil. HCl (1M) and extracted with methylene chloride. The organic layer was washed, dried over Na₂SO₄, and evaporated under reduced pressure to yield the crude residue which was purified with silica gel column chromatography CHCl₃/MeOH (98:2). The pure compound (Compound (6)) was isolated after concentration and drying of fractions as a white solid (51 mg, yield: 78%). ¹H-NMR: (CDCl₃) δ 1.14 (m, 2H, cyclopropyl-CH₂), 1.32 (m, 2H, cyclopropyl-CH₂), 1.39-1.49 (m, 6H, 3×CH₂), 1.49 (s, 9H, 3×CH₃), 1.58 (dt, J=6 Hz, 128 Hz, 2H, CH₂), 1.74 (dt, J=6, 12 Hz, 2H, CH₂), 3.22 (t, J=6 Hz, 4H, 2×CH₂), 3.43 (m, 1H, CH), 3.45-3.51 (m, 4H, piperidyl 2×CH₂), 3.62 (m, 2H), 3.63-3.67 (m, 4H, 2×CH₂), 7.31 (d, J=6 Hz, 1H, ArH), 8.03 (d, J=12 Hz, 1H, ArH), 8.81 (s, 1H, ArH), 10.1 (brs, 1H, NH). ¹³C-NMR: (CDCl₃) d 8.2, 25.4, 26.8, 28.4, 29.5, 32.6, 34.7, 39.2, 45.1, 50.0, 70.0, 70.2, 70.6, 71.3, 105.0, 111.4, 112.7, 112.8, 122.2, 138.4, 144.8, 146.8, 152.6, 154.2, 154.6, 165.1, 175.4.

### Synthesis of Compound (7)

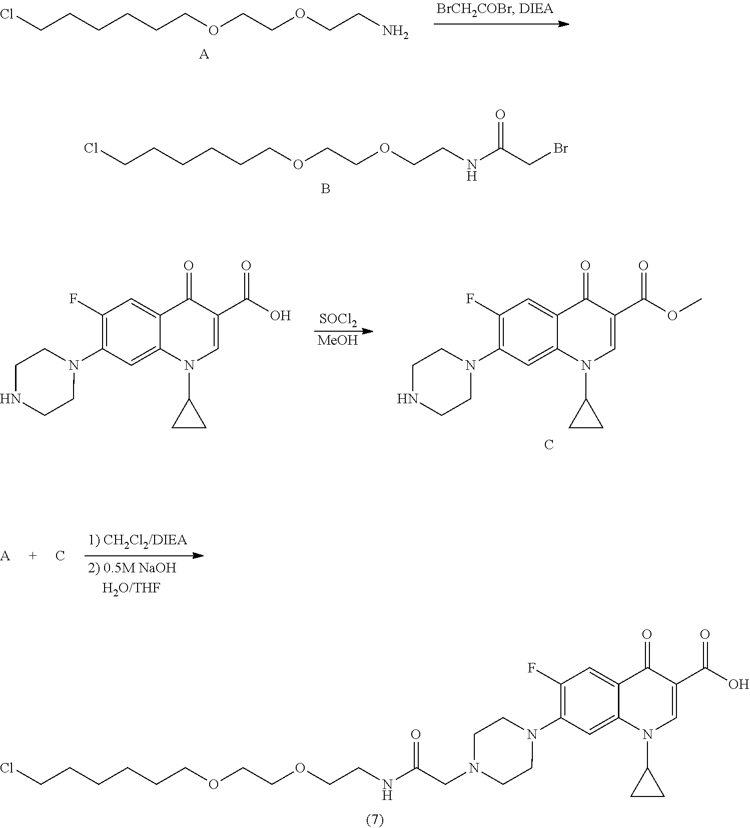

Compound (7). B: A solution of HaloTag amine ligand (26.0 mg, 0.1 mmol) was dissolved in 2.0 mL of dry methylene chloride. The solution was cooled on an ice water bath, and to this solution was added diisopropylethylamine (61 μL, 46.5 mg, 0.36 mmol). In another vial, bromoacetyl bromide (40.0 mg, 0.2 mmol) was dissolved in 1.0 mL methylene chloride and cooled in an ice bath, and to the bromoacetyl bromide solution was added (50 μL, 0.36 mmol) of triethylamine via a syringe. This mixture was stirred for 30 minutes, and it was then added to an ice-cold water solution of the HaloTag amine ligand prepared as described above. The reaction mixture was stirred on an ice water bath for 30 minutes, allowed to warm to room temperature, and was further stirred for 1 hr and concentrated under reduced pressure. The crude residue was diluted with 15 mL of ether, and to this, 10% NaHCO₃ (5.0 mL) was added, stirred for 5 min and transferred to a separatory funnel to remove the aqueous layer. The organic ether layer was washed with dilute HCl, followed by sat. NaHCO₃. The ether layer was finally washed with brine, dried over Na₂SO₄, concentrated, and used as is for the next reaction (31.0 mg).

Synthesis of ciprofloxacin methyl ester, C was carried out according to a reported procedure.¹ The compound was characterized by mass spectroscopy and ¹H-NMR spectroscopy. MF: C₁₉H₂₂FN₃O₃, Calc. 346.15; Obs. 346.1 for [M+H]⁺. ¹H-NMR: (CDCl₃) δ 1.14 (m, 2H, cyclopropyl-CH₂), 1.32 (m, 2H, cyclopropyl-CH₂), 3.12 (m, 4H, piperidyl 2×CH₂), 3.27 (m, 4H, piperidyl 2×CH₂), 3.43 (m, 1H, cyclopropyl CH—N), 3.91 (s, 3H, OCH₃), 7.26 (d, J=6 Hz, 1H, ArH), 8.02 (d, J=18 Hz, 1H, ArH), 8.54 (s, 1H, ArH).

The reaction of bromoacetyl-HaloTag amide with ciprofloxacin methyl easter, A+C: The ciprofloxacin methyl ester (18.0 mg, 0.052 mmol) was dissolved in methylene chloride and to this, bromeacetyl-HaloTag amide (25.0 mg, 0.072 mmol, 1.4 eq) was added followed by 40 μL of DIEA (0.23 mmol). The mixture was stirred overnight at room temperature and the reaction was analyzed by TLC. A new spot having fluorescence was observed (more non-polar compared with the starting ciprofloxacin methyl ester). The compound was purified by silica gel column chromatography using hexane/ethyl acetate (1:1) as eluent. The homogenous fractions as observed by TLC were combined and concentrated to afford ciprofloxacin-N-alkylated HaloTag methyl ester (23.0 mg, yield: 73%). ¹H-NMR: (CDCl₃) δ 0.92 (dd, J=Hz, 2H, Cyclopropyl CH₂), 1.20-1.39 (m, 18H), HRMS: for MF C₃₀H₄₃ClFN₄O₆. Calc. 609.2850; Obs. 609.2846 for [M+H]*.

Hydrolysis of the methyl ester to afford Compound (7): The ciprofloxacin-N-alkylated HaloTag amide (20.0 mg, 0.033 mmol) was dissolved in THF (5.0 mL) and to this was added 1.0M NaOH (200 μL). The mixture was vigorously stirred at room temperature for 4 hrs. TLC analysis showed that the methyl ester was consumed to form a new non-polar compound. The reaction mixture was concentrated under reduced pressure, acidified with dil. HCl (1M) and extracted with methylene chloride. The organic layer was washed, dried over Na₂SO₄, and evaporated under reduced pressure to yield the crude residue which was purified with silica column chromatography using (CHCl₃/MeOH; 98:2) as eluent. Homogenous fractions as observed by TLC were combined and concentrated under reduced pressure to yield a white solid (14.5 mg, yield: 74%). ¹H-NMR: (CDCl₃) δ 1.14 (m, 2H, cyclopropyl-CH₂), 1.32 (m, 2H, cyclopropyl-CH₂), 1.39-1.49 (m, 6H, 3×CH₂), 1.49 (s, 9H, 3×CH₃), 1.58 (dt, J=6 Hz, 128 Hz, 2H, CH₂), 1.74 (dt, J=6, 12 Hz, 2H, CH₂), 3.22 (t, J=6 Hz, 4H, 2×CH₂), 3.43 (m, 1H, CH), 3.45-3.51 (m, 4H, piperidyl 2×CH₂), 3.62 (m, 2H), 3.63-3.67 (m, 4H, 2×CH₂), 7.31 (d, J=6 Hz, 1H, ArH), 8.03 (d, J=12 Hz, 1H, ArH), 8.81 (s, 1H, ArH), 10.1 (brs, 1H, NH). ¹³C-NMR: (CDCl₃) d 8.2, 25.4, 26.8, 28.4, 29.5, 32.6, 34.7, 39.2, 45.1, 50.0, 70.0, 70.2, 70.6, 71.3, 105.0, 111.4, 112.7, 112.8, 122.2, 138.4, 144.8, 146.8, 152.6, 154.6, 165.1, 175.4. HRMS: for MF; C₂₉H₄₀ClFN₄O₆ Calc. 595.2699; Obs. 596.2690 for [M+H]⁺.

### Synthesis of Compound (8)

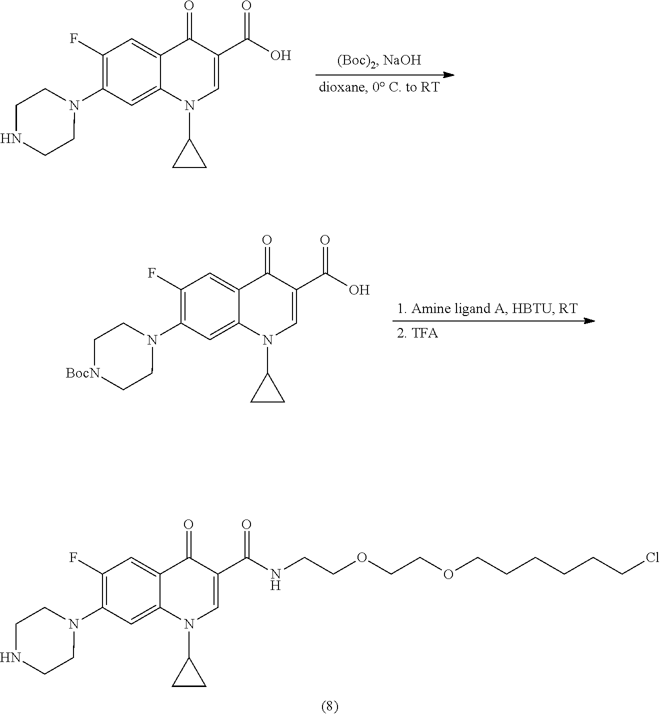 [SMILES: O=C(O)c1cn(C2CC2)c2cc(N3CCNCC3)c(F)cc2c1=O ; CC(C)(C)OC(=O)N1CCN(c2cc3c(cc2F)c(=O)c(C(=O)O)cn3C2CC2)CC1 ; O=C(NCCOCCOCCCCCCCl)c1cn(C2CC2)c2cc(N3CCNCC3)c(F)cc2c1=O]

Compound (8). N-^{t}Boc-Ciprofloxacin Synthesis: The compound was synthesized using a reported procedure with slight modifications.² Briefly, ciprofloxacin (330.0 mg, 1 mmol) was suspended in 25 mL of 1,4-dioxane. 10 mL of 1.0 M NaOH was added to the suspension and the mixture was stirred on an ice bath for 30 minutes prior to addition of di-tert-butyl dicarbonate (250 mg, 1.15 mmol). The mixture was allowed to warm up to room temperature and stirred overnight. Concentration of volatiles under reduced pressure using a rotary evaporator afforded a white solid which was filtered, washed with ice-cold water and with ether. The solid was dried in air to yield 7-(4-(tert-butoxycarbonyl) piperazin-1-yl)-1-cyclopropyl-6-fluoro-4-oxo-1,4-dihydroquionoline-3-carboxylic acid (375.0 mg. 87%). ¹H-NMR and mass spectral data matched with the reported compound.

Reaction of N-^{t}Boc-ciprofloxacin with HaloTag amine ligand, synthesis of N-^{t}Boc-ciprofloxacin-carboxy HaloTag amide: N-^{t}Boc-ciprofloxacin (86.2 mg, 2 mmol) was dissolved in 2.0 mL anhydrous DMF and to this was added DIEA (50 μL) followed by HBTU (100.0 mg, 2.6 mmol). After stirring for 1 hr at room temperature, a mixture of HaloTag amine (60.0 mg, 2.3 mmol) and DIEA (100 μL) in 0.5 mL DMF was added to the solution and further stirred for 4 hrs. TLC analysis indicated complete loss of the starting material to form a non-polar compound. Dichloromethane (25 ml) was added to the reaction mixture along with 15.0 mL of water transferred to a separatory funnel. The dichloromethane layer was isolated and washed with dil. HCl, water, and finally with brine. The organic layer was then dried over Na₂SO₄ and concentrated under reduced pressure yielding a residue which was further purified with flash chromatography over silica gel (CHCl₃/MeOH, 98:2). The homogenous fractions were collected and concentrated to yield a white solid (98.0 mg, yield: 77%). ¹H-NMR: (CDCl₃) δ 1.14 (m, 2H, cyclopropyl-CH₂), 1.32 (m, 2H, cyclopropyl-CH₂), 1.39-1.49 (m, 6H, 3×CH₂), 1.49 (s, 9H, 3×CH₃), 1.58 (dt, J=6 Hz, 128 Hz, 2H, CH₂), 1.74 (dt, J=6, 12 Hz, 2H, CH₂), 3.22 (t, J=6 Hz, 4H, 2×CH₂), 3.43 (m, 1H, CH), 3.45-3.51 (m, 4H, piperidyl 2×CH₂), 3.62 (m, 2H), 3.63-3.67 (m, 4H, 2×CH₂), 7.31 (d, J=6 Hz, 1H, ArH), 8.03 (d, J=12 Hz, 1H, ArH), 8.81 (s, 1H, ArH), 10.1 (brs, 1H, NH). ¹³C-NMR: (CDCl₃) d 8.2, 25.4, 26.8, 28.4, 29.5, 32.6, 34.7, 39.2, 45.1, 50.0, 70.0, 70.2, 70.6, 71.3, 105.0, 111.4, 112.7, 112.8, 122.2, 138.4, 144.8, 146.8, 152.6, 154.6, 165.1, 175.4.

Deprotection of N-tBoc-ciprofloxacin-carboxy HaloTag amide: N-′Boc-ciprofloxacin-carboxy HaloTag amide (60.0 mg, 0.094 mmol) was dissolved in 3.0 mL of dichloromethane in a 20 mL RB flask and stirred on an ice bath, and to this was added 0.5 mL of TFA. The mixture was stirred for 1 hr, during which time TLC analysis indicated complete consumption of the starting material. The reaction mixture was concentrated under reduced pressure and dichloromethane (5.0 mL) was added and evaporated. This dichloromethane cycle was repeated one more time to remove TFA to afford Ciprofloxacin-carboxy-HaloTag amide TFA salt as Compound (8). Trituration with ether gave a white solid (57.0 mg). ¹H-NMR: (CDCl₃) δ 1.17 (m, 2H, cyclopropyl-CH₂), 1.34-1.42 (m, 4H, cyclopropyl-CH₂ & CH₂), 1.43 (m, 2H, CH₂), 1.58 (dt, J=6 Hz, 128 Hz, 2H, CH₂), 1.73 (dt, J=6, 12 Hz, 2H, CH₂), 3.16 (m, CH₂), 3.45-3.55 (m, 12H, CH₂), 3.55 (m, 2H, CH₂), 3.60-3.69 (m, CH₂), 7.37 (d, J=6 Hz, 1H, ArH), 8.04 (d, J=12 Hz, 1H, ArH), 8.82 (s, 1H, ArH), 10.1 (brs, 1H, NH). ¹³C-NMR: (CDCl₃) d 8.3, 25.5, 26.8, 29.6, 32.7, 34.9, 39.3, 43.6, 45.2, 47.1, 50.0, 70.0, 70.2, 70.6, 71.4, 111.6, 113.1, 123.3, 138.4, 143.4, 143.5, 147.2, 152.5, 154.1, 175.3.

### Biochemical Methods

For all biological assays, laboratory strains of wild type E. coli (ATCC 25922), and K12 E. coli strains BL21(DE3) and E. coli Lemo21(DE3) were grown with shaking at 37° C. overnight from freezer glycerol stocks in 5 mL of Luria Broth (LB) media.

HaloTag Protein Expression: Cytoplasmic HaloTag protein. For expression of the HaloTag protein in E. coli, HaloTag expression plasmid Cyto_Halo_pET21(b)+ was transformed into E. coli BL21(DE3) and grown on an LB/agar plate with ampicillin (100 μg/mL) at 37° C. overnight. Colonies were picked and grown in 5 mL LB broth with ampicillin at 37° C. overnight. A culture tube containing 2 mL LB media was inoculated with the overnight culture at a ratio of 1:100 and cells were grown at 37° C. for 3 h hours, or until the optical density at 600 nm reached 0.6. Cultures were induced with 1 mM Isopropyl β-d-1-thiogalactopyranoside (IPTG) at 37° C. for 2 hours in a shaker incubator, and then pelleted at 4,000 rpm for 3 min in an HERAEUS Multicentrifuge X1 centrifuge (Thermofisher Scientific). The pellet was washed three times with 1×PBS pH 7.2 following which cells were ready for assaying. The identity of the protein was confirmed by SDS-PAGE.

HaloTag Protein Expression: Periplasmic HaloTag protein. For localization of HaloTag protein in the periplasm, a modification of Cyto_Halo containing an amine terminal recognition sequence, DsbA for export to the periplasm by the SecA pathway, i.e., Peri_Halo_pET21(b)+ was transformed into E. coli Lemo21(DE3) and grown on an LB/agar plate with ampicillin (100 μg/mL) and chloramphenicol (34 μg/mL) at 37° C. overnight. Colonies were picked and grown in 5 mL LB broth with ampicillin/chloramphenicol at 37° C. overnight. A culture tube containing 2 mL LB media was inoculated with the overnight culture at a ratio of 1:100 and cells were grown at 37° C. for 3 hrs, or until the optical density at 600 nm reached 0.6. Cultures were induced with 1 mM Isopropyl β-d-1-thiogalactopyranoside (IPTG) at 37° C. for 2 hours in a shaker incubator, and then pelleted at 4,000 rpm for 3 min in an HERAEUS Multicentrifuge X1 centrifuge (Thermofisher Scientific). The pellet was washed three times with 1×PBS pH 7.2 following which cells were ready for assaying. The identity of the protein was confirmed by SDS-PAGE.

BaCAPA Pulse-Chase Protocol (chloroalkane-tagged molecule, chloroalkane-tagged dye). For a bacterial chloroalkane penetration assay (BaCAPA), 25 μL of serially diluted samples of the chloroalkane-tagged test molecule, in quadruplicate, were transferred into a 96-well plate at 2× of the desired concentration. The pellet obtained from the washing step above was resuspended in 2 mL of 1×PBS by vortexing thoroughly. 25 μL of the cell suspension (both IPTG induced and non-induced cells was added into the wells and incubated at 37° C. with shaking at 250 RPM. After 30 min, the plate was centrifuged to pellet the cells using a Jouan C4i centrifuge (Thermofisher Scientific). The supernatant was discarded, and the pellet was washed twice with PBS. The chase step followed by addition into each test well, 50 μL of 5 μM chloroalkane-tagged rhodamine 110 and incubated for 30 min at 37° C. with shaking in a shaker incubator at 250 RPM. The plate was centrifuged to pellet the cells and cells were washed three times with PBS and fixed with 4% formaldehyde by incubating at room temperature for 30 min. The cells were analyzed by flow cytometry on the Attune NxT Acoustic Focusing Cytometer (Invitrogen) by exciting using the blue laser (530 nm).

BaCAPA Control Experiment: pulsing with a chloroalkane-tagged dye. 25 μL of the cell suspension (both IPTG induced and non-induced cells) was added into the wells of a 96-well plate. The plate was centrifuged to pellet the cells using a Jouan C4i centrifuge (Thermofisher Scientific). The supernatant was discarded and into each test well, 50 μL of chloroalkane-tagged rhodamine 110 was added and incubated for 30 min at 37° C. in a shaker incubator (250 RPM). The plate was centrifuged to pellet the cells and cells were washed three times with PBS and fixed with 4% formaldehyde by incubating at room temperature for 30 min. The cells were analyzed by flow cytometry on the Attune NxT Acoustic Focusing Cytometer (Invitrogen) by exciting using the blue laser (530 nm).

Polymyxin nonapeptide (PMBN) mediated bacterial chloroalkane-tagged molecule penetration. For the PMBN-mediated penetration assay, 25 μL of the desired PMBN concentration was added into the wells of a 96-well plate at 3× the final concentration along with 25 μL of the HaloTag protein cell suspension (both IPTG induced and non-induced cells). Next, 25 μL of the serially diluted samples of the chloroalkane-tagged test molecule was added into the wells at 3× the desired final concentration, in quadruplicate. The plates were incubated at 37° C. with shaking at 250 RPM. After 30 min, the plate was centrifuged to pellet the cells using a Jouan C4i centrifuge (Thermofisher Scientific). The supernatant was discarded, and the pellet was washed twice with PBS. The chase step followed by addition into each test well, 50 μL of 5 mM chloroalkane-tagged rhodamine 110 and incubated for 30 min at 37° C. with shaking in a shaker incubator at 250 RPM. The plate was centrifuged to pellet the cells and cells were washed three times with PBS and fixed with 4% formaldehyde by incubating at room temperature for 30 min. The cells were analyzed by flow cytometry on the Attune NXT Acoustic Focusing Cytometer (Invitrogen) by exciting using the blue laser (530 nm).

Mammalian Cell Culture. J774A. 1 cells were cultured in Dulbecco's modified Eagle's medium (DMEM) supplemented with 10% (v/v) FBS, 50 IU/mL penicillin, 50 μg/mL streptomycin, and 2 mM L-glutamine in a humidified atmosphere of 5% CO₂ at 37° C.

BaCAPA labeling of E. coli inside macrophages. J774A. 1 macrophages were cultured to confluency, resuspended in antibiotic free media (DMEM+10% FBS containing no Pen-Strep), and were added to a 96-well plate with 200,000 cells/well. The cells were centrifuged at 1,000 rpm for 5 mins to pellet the cells. The macrophage pellets were resuspended in antibiotic free media containing HaloTag protein expressing E. coli BL21(DE3) (either IPTG induced or non-induced) at a MOI 50 and incubated at 37° C. for 15 mins to promote bacterial cell uptake into macrophages. The macrophages were pelleted, resuspended in DMEM+10% FBS+300 μg/mL gentamycin (to clear non-phagocytosed bacteria)+5 μM of chloroalkane-tagged rhodamine 110 and incubated for 5 min at 37° C. The macrophages were washed 3× with 1×PBS to remove excess dye and cells were fixed for 10 min at 4° C. with 4% formaldehyde in 1×PBS. Cells were analyzed using the Attune NxT Flow Cytometer (Thermo Fischer) equipped with a 488 nm laser and 525/40 nm bandpass filter. Cells were gated to analyze only the population of macrophages. For confocal microscopy, J774A macrophages were treated with 5 μg/mL of TMR-tagged Wheat Germ Agglutinin (Vector Laboratories, RL-1022) for 30 min at 4° C. Glass microscope slides were spotted with a 1% agar pad and 5 μL of sample were deposited onto the agar. Samples were covered with a micro cover glass and imaged using a Zeiss 880/990 multiphoton Airyscan microscopy system (40× oil-immersion lens) equipped with 488 nm and 550 nm lasers. Images were obtained and analyzed via Zeiss Zen software. We acknowledge the Keck Center for Cellular Imaging and for the usage of the Zeiss 880/980 multiphoton Airyscan microscopy system (PI-AP: NIH-OD025156).

BaCAPA labeling of E. coli for confocal microscopy imaging. 25 μL of the cell suspension of HaloTag protein expressing E. coli BL21(DE3) (either IPTG induced or non-induced) was added into the wells of a 96-well plate. The plate was centrifuged to pellet the cells using a Jouan C4i centrifuge (Thermofisher Scientific). The supernatant was discarded and into each test well, 50 μL of chloroalkane-tagged rhodamine 110 was added and incubated for 30 min at 37° C. in a shaker incubator (250 RPM). The plate was centrifuged to pellet the cells and cells were washed three times with PBS and fixed with 4% formaldehyde by incubating at room temperature for 30 min. The cells were analyzed by flow cytometry on the Attune NXT Acoustic Focusing Cytometer (Invitrogen) by exciting using the blue laser (530 nm). For confocal microscopy, glass microscope slides were spotted with a 1% agar pad and 2 μL of fixed bacterial samples were deposited onto the agar. Samples were covered with a micro cover glass and imaged using a Zeiss 880/990 multiphoton Airyscan microscopy system (60× oil-immersion lens) equipped with a 488 nm laser. Images were obtained and analyzed via Zeiss Zen software. We acknowledge the Keck Center for Cellular Imaging and for the usage of the Zeiss 880/980 multiphoton Airyscan microscopy system (PI-AP: NIH-OD025156).

Minimum Inhibitory Concentration. Compounds were serially diluted 2-fold from stock solutions to yield 12 test concentrations in U-bottom, 96-well plates. Overnight cultures were used to inoculate LB media 1:100 and regrown to exponential phase, as determined by optical density recorded at 600 nm (OD₆₀₀). All cultures were diluted again to ca. 10⁶ CFU/mL in MH media, and 100 μL was inoculated into each well of a U-bottom 96-well plate (BD Biosciences, BD 351177) containing 100 μL of compound solution (in line with CLSI Standards).⁴Plates were incubated statically at 37° C. for 24 h upon which time wells were evaluated visually for bacterial growth. The MIC was determined as the lowest concentration of compound resulting in no bacterial growth visible to the naked eye, based on the majority of three independent experiments. Growth and sterility controls were conducted for each plate.

### Example A: Bacterial Azide Permeability Assay, BAPA

The purpose of this example is to demonstrate synthesis of a chloroalkane-DBCO and, using the BAPA workflow, to demonstrate that cellular fluorescence levels increased with increasing concentration of IPTG (FIG. 8A). After induction with IPTG, cells were treated with azide-modified Rhodamine 110 (R110-N3) and cellular fluorescence was measured by flow cytometry. There was observed higher fluorescence levels with increasing concentration of R110-N₃ (FIG. 8B). Other controls were also performed, including the measurement of cellular fluorescence levels in the absence of chloroalkane-DBCO and in the presence of rhodamine without the azide handle. Additional assay parameters were evaluated including incubation times, working buffers, and dye character. Through these empirically defined parameters, a set of assay conditions were reached, and these conditions were used to test a small panel of azide-modified molecules using azide reagents shown in Table 1 above (FIG. 8C).

### Example B: Targeted Library of Antibiotic Compounds

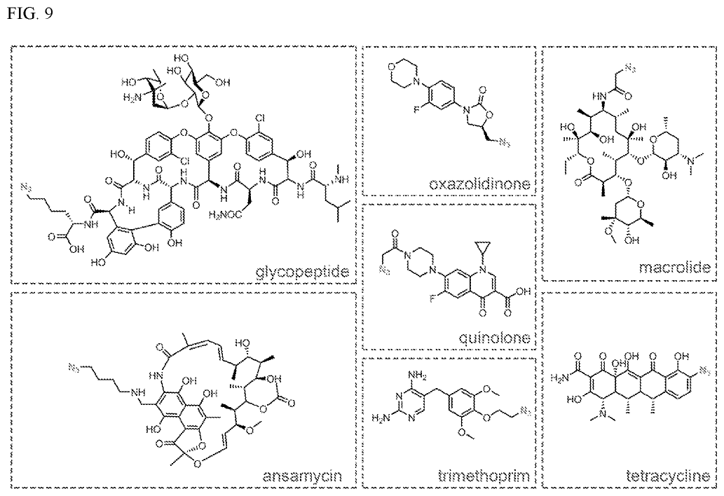

The purpose of this example is to illustrate azide tagging on exemplary antibiotic compounds. Seven derivatives of known antibiotics were synthesized with each displaying an azide tag while aiming to minimally disrupt their biological activity (FIG. 9). All compounds were fully characterized using ¹H, ¹³C NMR, high resolution mass spectrometry and analytical RP-HPLC to determine purity.

### Example C: Large Scale Library

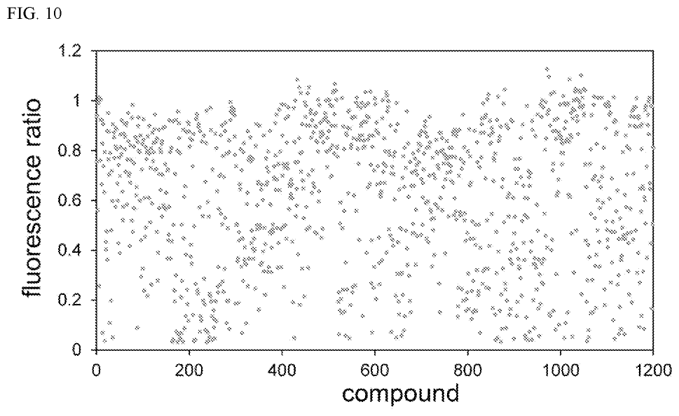

The purpose of this example is to demonstrate analysis of a highly diverse library of azide tagged small molecules using BAPA. A modular synthetic method to add an azide tag to small molecules generated a 1224-member library in 96-well plates (Meng G, Guo T, Ma T, et al. Modular click chemistry libraries for functional screens using a diazotizing reagent (Nature. October 2019; 574(7776):86-89). The method has expanded the library to 5000 molecules, which is fully validated and dispensed in 96-well plates. These molecules are structurally unique, cover a wide range of chemical space, and span a range of molecular weights (200-500 Da). In an exemplary assay using the process described herein, a pilot screen was performed in E. coli using a sub-panel of about 1200 azide-conjugated compounds (FIG. 10). The results demonstrated that the process described herein revealed the permeation of a variety of compounds into E. coli.

Numbered references in the Examples section are as follows:
1. Shahzad S. A. et al. Bioorganic Chemistry, 100, 103076, 2020.
2. Chen J, Su F Y, Das D, Srinivasan S, Son H N, Lee B, Radella F 2nd, Whittington D, Monroe-Jones T, West T E, Convertine A J, Skerrett S J, Stayton P S, Ratner D M. Glycan targeted polymeric antibiotic prodrugs for alveolar macrophage infections. Biomaterials. 2019 March; 195:38-50.
3. Uma N. Sundram and John H. Griffin. General and Efficient Method for the Solution- and Solid-Phase Synthesis of Vancomycin Carboxamide Derivatives. The Jornal of Organic Chemistry. 1995, 60, 5, 1102-1103
4. Al-Khalifa, S. E., Jennings, M. C., Wuest, W. M., and Minbiole, K. P. (2017) The Development of Next-Generation Pyridinium-Based multiQAC Antiseptics. ChemMedChem 12, 280-283)

All patent and non-patent references cited herein are incorporated in their entireties as if fully set forth herein.

1. A process for determining whether a small molecule is a potential antibiotic drug, comprising:
(a) contacting the small molecule that has been linked to a chloroalkane moiety with a bacteria that expresses a mutant form of bacterial haloalkane dehalogenase to yield a first population of treated bacteria;
(b) contacting the first population of treated bacteria with a fluorophore that is linked to a chloroalkane moiety to produce a second population of treated bacteria;
(c) detecting the fluorescence signal of the second population of treated bacteria; and
(d) determining that a low fluorescence signal relative to background fluorescence correlates to a conclusion of high accumulation of the small molecule within the bacteria, and that a high fluorescence signal relative to background fluorescence correlates to a conclusion of low accumulation of the small molecule within the bacteria.

2. The process according to claim 1, wherein the fluorophore is a rhodamine dye or coumarin.

3. The process according to claim 2, wherein the fluorophore is a rhodamine dye.

4. The process according to claim 1, wherein the chloroalkane moiety linked to the fluorophore is of the formula: 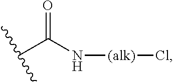
wherein alk is a straight or branched C₁-C₂₀-alkyl optionally interrupted by 1 to 6 oxygen atoms.

5. The process according to claim 1, wherein the chloroalkane moiety linked to the small molecule is of the formula: 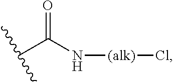
wherein alk is a straight or branched C₁-C₂₀-alkyl optionally interrupted by 1 to 6 oxygen atoms.

6. The process according to claim 4 or 5, wherein alk is a C₁₀-C₁₅-alkyl optionally interrupted by 1 to 3 oxygen atoms.

7. The process according to claim 6, wherein alk is of the formula:

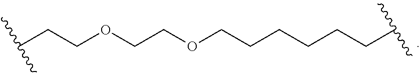 [SMILES: *CCCCCCOCCOCC*]

8. A process for determining whether a small molecule is a potential antibiotic drug, comprising:
(a1) contacting a bacteria that expresses a mutant form of bacterial haloalkane dehalogenase with a reagent of the general formula (I) to yield a tagged population of bacteria:
[strained alkyne]-(linker)-(alk-Cl) (1)
wherein
[strained alkyne] is a moiety containing a strained alkyne functional group;
(linker) is a cyclic linker group, linear linker group, or a combination thereof; and
(alk-Cl) is a straight or branched C₁-C₂₀-chloroalkyl optionally interrupted by 1 to 6 oxygen atoms,
(b1) contacting the tagged population of bacteria with the small molecule that has been linked to an azide moiety to yield a first population of treated bacteria;
(c1) contacting the first population of treated bacteria with a fluorophore that is linked to an azide moiety to yield a second population of treated bacteria;
(d1) detecting a fluorescence signal of the second population of treated bacteria; and
(e1) determining that a low fluorescence signal relative to background fluorescence correlates to a conclusion of high accumulation of the small molecule within the bacteria, and that a high fluorescence signal relative to background fluorescence correlates to a conclusion of low accumulation of the small molecule within the bacteria.

9. The process according to claim 8, wherein (linker) is selected from a bond, amino, amido, carboxyl, C₁-C₆-ester, C₁-C₆-alkoxy, linear and cyclic boronic esters, 3- to 8-membered heterocycloalkyl (wherein 1 to 3 heteroatoms are selected from N, O, and S), C₃-C₈-cycloalkyl optionally fused to 1 or 2 C₆-C₁₀-aryl, C₆-C₁₀-aryl, 5- to 10-membered heteroaryl (wherein 1 to 4 heteroatoms are selected from N, O, and S), succinimidyl, C₁-C₆-alkyl optionally interrupted by one or more of —C(O)— and —N(H)—, and combinations thereof.

10. The process according to claim 8, wherein the fluorophore is a rhodamine dye or coumarin.

11. The process according to claim 10, wherein the fluorophore is a rhodamine dye.

12. The process according to claim 8, wherein [strained alkyne] comprises a C₇-C₁₄-cycloalkyne moiety.

13. The process according to claim 12, wherein [strained alkyne] is dibenzocyclooctyne (DBCO).

14. The process according to claim 8, wherein alk-Cl is a C₁₀-C₁₅-chloroalkyl optionally interrupted by 1 to 3 oxygen atoms.

15. The process according to claim 14, wherein alk-Cl is of the formula:

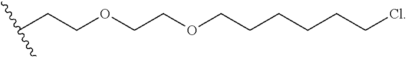 [SMILES: *CCOCCOCCCCCCCl]

16. The process according to claim 1 or 8, wherein the bacteria is gram-negative bacteria.

17. The process according to claim 16, wherein the bacteria is E. coli.
